(19) United States
(12) Patent Application Publication
(10) Pub. No.: US 2008/0108057 A1
(43) Pub. Date: May 8, 2008

(54) Allelic imbalance in the diagnosis and prognosis of cancer

(76) Inventors: Jeffrey K. Griffith, Cedar Crest, NM (US); Christopher M. Heaphy, Albuquerque, NM (US); Marco Bisoffi, Albuquerque, NM (US); William C. Hines, Albuquerque, NM (US)

Correspondence Address: MUETING, RAASCH & GEBHARDT, P.A., P.O. BOX 581415, MINNEAPOLIS, MN, 55458, US

(21) Appl. No.: 11/158,653

(22) Filed: Jun. 22, 2005

Related U.S. Application Data
(60) Provisional application No. 60/624,248, filed on Nov. 2, 2004.
(60) Provisional application No. 60/581,928, filed on Jun. 22, 2004.

(51) Int. Cl. C12Q 1/68

(52) U.S. Cl. 435/6

(57) ABSTRACT

Methods for assessing the extent of allelic imbalance in a genomic nucleic acid sample. Methods for diagnosing cancer and determining the prognosis of a patient with cancer, including breast or prostate cancer, by assessing the extent of allelic imbalance in a genomic nucleic acid sample.

This application claims the benefit of U.S. Provisional Application Ser. Nos. 60/581,928, filed Jun. 22, 2004, and 60/624,248, filed Nov. 2, 2004, each of which is incorporated by reference herein.

### GOVERNMENT FUNDING

The present invention was made with government support under Grant Nos. DAMD17-98-1-85333, DAMD17-01-1-0572, DAMD17-00-1-0370 and DAMD17-02-1-0514, awarded by the Department of Defense, Breast Cancer Research Program; Grant Nos. R25 GM60201 and T34 GM08751, awarded by the National Institutes of Health; and Grant No. R33 CA86136, awarded by the National Cancer Institute, National Institutes of Health. The Government may have certain rights in this invention.

### BACKGROUND

Cancer is a genetic disease, arising from an accumulation of mutations that promote the selection of cells with increasingly malignant phenotypes. Previous studies have shown that a driving force behind this process is genomic instability, which is a hallmark of cancer cells. While genomic instability is an important factor in the pathogenesis and progression of human cancers, the precise molecular mechanisms underlying genomic instability, such as chromosomal rearrangements, remain largely unknown (Gollin, Curr Opin Oncol 2004, 16:25-31; Charames and Bapat, Curr Mol Med 2003, 3:589-596; Nojima, Methods Mol Biol 2004, 280:3-49; and Lengauer et al., Nature 1998, 396:643-649). Thus, there exists a need for improved methods to assess the extent of genomic instability in cancer cells and tumors.

Although mechanistic insights into the molecular pathology of cancer are increasing, the question of how carcinogenesis is initiated in human tissues remains largely unanswered. The concepts of “field cancerization” and “cancer field effect” have been introduced to describe areas within tissues consisting of histologically normal, yet genetically aberrant, cells that represent fertile grounds for tumorigenesis. Slaughter and colleagues first introduced the concept of “field cancerization” in 1953 to explain the multifocal and independent areas of histologically pre-cancerous alterations occurring in oral squamous cell carcinomas (Slaughter et al., Cancer 1953, 6:963-968; reviewed by Braakhuis et al., Cancer Res 2003, 63:1727-1730; and Garcia et al., J Pathol 1999, 187:61-81). Organ systems in which field cancerization has been implied include lung, colon, cervix, bladder, skin, and breast (Hockel and Dornhofer, Cancer Res 2005, 65:2997-3002).

Previous investigators have reported that genetic alterations occur in histologically normal tissues adjacent to breast tumors (Aubele et al., Diagn Mol Pathol 2000, 9:14-19; Farabegoli et al., J Pathol 2002, 196:280-286; Deng et al., Science 1996, 274:2057-2059; Forsti et al., European J Cancer 2001, 37:1372-1380; Lakhani et al., Journal of Pathology 1999, 189:496-503; Larson et al., Am J Pathol 2002, 161:283-290; Meeker et al., Am J Pathol 2004, 164:925-935; Euhus et al. Journal of the National Cancer Institute 2002, 94:858-860; and Ellsworth et al., Breast Cancer Res Treat 2004, 88:131-139). Such fields of genomic instability that support tumorigenic events have important clinical implications. First, such fields can give rise to clonal selection of precursor cells that ultimately lead to the development of cancer (Ellsworth et al., Lancet Oncol 2004, 5:753-758). Second, the presence of such fields, even after surgical resection of primary tumors, represents a continuous risk factor for cancer recurrence or formation of secondary lesions (Garcia et al., J Pathol 1999, 187:61-81; and Li et al., Cancer Res 2002, 62:1000-1003). Thus, there exists a need for methods to better define the extent and spatial distribution of genoric instability in tissues adjacent to tumors. Such methods would be of practical importance in the identification of tumor margins, the assessment of recurrence risk factors, and the consideration of tissue-sparing surgery.

In most cancers, currently available prognostic markers fail to differentiate between aggressive tumors and comparatively indolent or non-aggressive tumors. This problem can be particularly acute with cancers such as breast and prostate cancers. For example, prognostic markers of breast cancer, including nodal status and tumor size, generally do not differentiate between aggressive tumors that have metastasized beyond the breast to the axial lymph nodes at the time of diagnosis and indolent tumors that have not metastasized. Accordingly, many women with breast cancer receive adjuvant chemotherapies and hormonal therapies that are, in many instances, unnecessary. Although adjuvant therapies improve overall survival, particularly of high-risk patients, the consequences and complications of these therapies, which include fatigue, nausea, vomiting, alopecia, myelosuppression, cardiotoxicity, and the development of secondary malignancies, including leukemia, are severe and markedly reduce the patients' quality of life. The same prognostic and therapeutic challenges are present with prostate cancer and other cancers. Thus, there exists a need for methods that reliably predict the likelihood of recurrence of a cancer so as to differentiate between the subsets of patients that will benefit from adjuvant therapy from those who can be spared unnecessary side effects.

### SUMMARY OF THE INVENTION

The present invention provides a method of detecting allelic imbalance in genomic nucleic acid, the method including amplifying a plurality of short tandem repeat (STR) loci in the genomic nucleic acid, wherein the STR loci are unlinked, and wherein each allele of each different STR locus yields an amplicon product; detecting the resultant amplicon products; and calculating an allelic ratio for each STR locus, wherein a statistically significant allelic ratio of greater than 1.0 for a STR locus indicates an allelic imbalance at the STR locus. In some embodiments, three or more STR loci exhibit allelic imbalance.

In another aspect, the present invention provides a method of determining cancer prognosis, the method including amplifying a plurality of short tandem repeat (STR) loci in a genomic nucleic acid sample from histologically normal, tumor-adjacent tissue, wherein the STR loci are unlinked, and wherein each allele of each different STR locus yields an amplicon product; detecting the resultant amplicon products; and calculating an allelic ratio for each STR locus, wherein a statistically significant allelic ratio of greater than 1.0 for a STR locus indicates an allelic imbalance at the STR locus, and wherein an allelic imbalance in at least one STR locus is indicative of a cancer with an increased risk for metastasis, recurrence and/or death. In some embodiments, three or more STR loci are amplified and allelic imbalance in at least three STR loci is indicative of a cancer with an increased high risk for metastasis, recurrence and/or death.

In another aspect, the present invention provides a method of identifying a tumor margin, the method including amplifying a plurality of short tandem repeat (STR) loci in a genomic nucleic acid sample from tumor-adjacent tissue, wherein the STR loci are unlinked, and wherein each allele of each different STR locus yields an amplicon product; detecting the resultant amplicon products; and calculating an allelic ratio for each STR locus, wherein a statistically significant ratio of greater than 1.0 for a STR locus indicates an allelic imbalance at the STR locus, and wherein an allelic imbalance in at least one STR locus identifies the tumor-adjacent tissue as within the margin of the tumor. In some embodiments, three or more STR loci are amplified and an allelic imbalance in at least three STR loci identifies the tumor-adjacent tissue as within the margin of the tumor.

In another aspect, the present invention provides a method of diagnosing cancer, the method including amplifying a plurality of short tandem repeat (STR) loci in a genomic nucleic acid sample, wherein the STR loci are unlinked, and wherein each allele of each different STR locus yields an amplicon product; detecting the resultant amplicon products; and calculating an allelic ratio for each STR locus, wherein a statistically significant allelic ratio of greater than 1.0 for a STR locus indicates an allelic imbalance at the STR locus, and wherein an allelic imbalance in at least one STR locus indicates that the sample includes cancerous cells. In some embodiments, three or more STR loci are amplified and allelic imbalance in at least three STR loci indicates that the sample includes cancerous cells.

In another aspect, the present invention provides a method of identifying a predisposition to cancer, the method including amplifying a plurality of short tandem repeat (STR) loci in a genomic nucleic acid sample from an individual with a suspected predisposition to cancer, wherein the STR loci are unlinked, and wherein each allele of each different STR locus yields an amplicon product, detecting the resultant amplicon products; and calculating an allelic ratio for each STR locus, wherein a statistically significant allelic ratio of greater than 1.0 for a STR locus indicates an allelic imbalance at the STR locus, and wherein an allelic imbalance in at least one STR locus indicates that the subject has a predisposition to cancer. In some embodiments, three or more STR loci are amplified and allelic imbalance in at least three STR loci indicates that the subject has a predisposition to cancer.

In the methods of the present invention, detecting the resultant amplicon products may be, for example, by electrophoretic separation and by mass spectrometry. Detecting the resultant amplicon products may be carried out in a single preparation or in more than one preparation.

In the methods of the present invention, an allelic ratio of 1.28 or greater, 1.37 or greater, 1.61 or greater, or 2.15 or greater may indicate allelic imbalance at a STR locus.

In the methods of the present invention, the genomic nucleic acid may be obtained, for example, from normal cells, tumor cells, including, but not limited to, breast cancer cells, prostate cancer cells, renal cancer cells, or endometrial cancer cells, and histologically normal cells adjacent to a tumor.

In the methods of the present invention, a plurality of STR loci may be amplified. For example, at least 12 different STR loci may be amplified and at least 16 different STR loci may be amplified.

In the methods of the present invention one or more of the STR loci amplified may be selected from amelogenin, CSF1PO, D2S1338, D3S1358, D5S818, D7S820, D8S1179, D13S317, D16S539, D18S51, D19S433, D2S11, FGA, TH01, TPOX, or vWA.

In the methods of the present invention the STR loci amplified may include amelogenin, CSF1PO, D2S1338, D3S1358, D5S818, D7S820, D8S1179, D13S317, D16S539, D18S51, D19S433, D21S11, FGA, TH01, TPOX, and vWA.

Unless otherwise specified, “a,” “an,” “the,” and “at least one” are used interchangeably and mean one or more than one.

### BRIEF DESCRIPTION OF THE FIGURES

FIGS. 1A-1C. Electropherograms of VIC-labeled amplicons from matched normal (FIG. 1A) and renal carcinoma (FIG. 1B) tissues. FIG. 1C presents the distribution of allelic peak height ratios in buccal cells. Only VIC-labeled amplicons are shown. The D3S1358, TH01 and D2S1338 loci are heterozygous and D13S317 and D16S539 loci are homozygous. Fluorescent intensity is shown on the Y-axis and amplicon size, in base pairs, is shown on the x-axis. The ratios of the fluorescent intensities of each allelic pair of heterozygous loci are shown. Loci with allelic ratios of 1.61 and greater are defined as sites of allelic imbalance and are displayed for matched normal (FIG. 1A) or tumor (FIG. 1B). A histogram of the peak height ratios of the 318 heterozygous alleles is displayed (FIG. 1C). A box/whisker plot is located above the histogram. The line across the middle of the box identifies the median sample value. The ends of the box are the 25th and 75th quartiles, and the difference between these quartiles (0.14) is the interquartile range (IQR). The IQR was used to compute the 1.61 definition for outliers.

FIG. 2. Frequency of allelic imbalance (AI) in normal and tumor cells. The numbers of sites of allelic imbalance (i.e. 0, 1, 2 or 3 or greater) were determined in 28 frozen samples of normal buccal cells, 10 frozen samples of normal renal tissue, 22 frozen samples of renal carcinomas, 46 frozen samples of breast carcinomas, 27 paraffin-embedded samples of breast carcinomas, and 31 paraffin-embedded samples of prostate carcinomas.

FIG. 3. Effect of admixtures of matched normal and renal carcinoma DNA on peak height ratios. The specified admixtures were generated using DNA from a matched pair of normal renal tissue and renal cell carcinoma. Data from the heterozygous D3S1358 locus is shown. The allelic ratios are 1.09 in the normal renal tissue and 2.02 in the renal carcinoma. The best-fit line was generated by linear regression and has a correlation coefficient (R 2) of 0.965.

FIG. 4. Relationship Between Telomere DNA Content and Allelic Imbalance in Prostate Tumors (Tumor Tissue) and Coexisting Histologically Normal Tissue (CHN Tissue). Telomere DNA content (TC) and allelic imbalance (AI) were measured in DNA purified from 31 prostate tumors and 27 coexisting histologically normal tissues prostate tissues. The box shows the group median as a line across the middle and the quartiles (25th and 75th percentiles) as its ends. The 10th and 90th quantiles are shown as lines above and below the box.

FIG. 5. Relationship Between Telomere DNA Content in Prostate Tumors (Tumor Tissue) and Coexisting Histologically Normal Prostate Tissue (CHN Tissue) and 72-month Recurrence-free Survival. Telomere DNA content (TC) was measured in 49 prostate tumors and 30 coexisting histologically normal tissues. The analysis included men with no recurrence within 72 months after prostatectomy and men with documented distant metastasis, biochemical recurrence (rising PSA) or death as a consequence of prostate cancer within 72 months after prostatectomy. The box shows the group median as a line across the middle and the quartiles (25th and 75th percentiles) as its ends. The 10th and 90th quantiles are shown as lines above and below the box.

FIG. 6. Recurrence-free Survival By Telomere DNA Content in Prostate Tumors. The cohort was divided into two groups, based on the specified values of telomere DNA content (TC). The prostate cancer-free survival interval, in months, is shown on the x-axis and the disease-free fraction is shown on the y-axis.

FIGS. 7A-7B. Telomere DNA content and extent of allelic imbalance in normal, tumor, and histologically normal, tumor-adjacent breast tissues. FIG. 7A represents telomere DNA content (TC) in 20 disease-free breast tissues (normal) and in 38 tumor and matched histologically normal, tumor-adjacent breast tissues, excised at unknown distances from the visible tumor margin. FIG. 7B represents extent of allelic imbalance (AI) in 20 disease-free breast tissues (normal) and in 23 tumor and matched histologically normal, tumor-adjacent breast tissues, excised at unknown distances from the visible tumor margin. TC is expressed as % of placental control multiplied by 10-2. Wilcoxon Kruskal/Wallis Rank Sums analyses (p values) at a significance level of 0.05 are indicated to compare TC and allelic imbalance between the different types of tissue. The diamonds indicate the group mean (line across middle) and the 95% confidence intervals (upper and lower lines). Although the data points are vertically shifted, some are still overlapping. The vertical dotted line separates the disease-free from the diseased breast tissues. “HN” represents histologically normal.

FIGS. 8A-8B. Telomere DNA content and extent of allelic imbalance as a function of distance, i.e. at 1 and 5 cm, from the visible tumor margins in 11 breast cancer cases, and in 20 disease-free breast tissues (normal). FIG. 8A represents telomere DNA content (TC) as a function of distance, i.e. at 1 and 5 cm, from the visible tumor margins. FIG. 8B represents the extent of allelic imbalance (AI) as a function of distance, i.e. at 1 and 5 cm, from the visible tumor margins. TC is expressed as % of placental control multiplied by 10-2. Wilcoxon Kruskal/Wallis Rank Sums analyses (p values) at a significance level of 0.05 are indicated to compare TC and AI between the different types of tissue. The diamonds indicate the group mean (line across middle) and the 95% confidence intervals (upper and lower lines). Although the data points are vertically shifted, some are still overlapping. The vertical dotted line separates the disease-free from the diseased breast tissues. “HN” indicates histologically normal.

FIG. 9. Recurrence-free survival by allelic imbalance in breast tumors. The cohort was divided into two groups, based on the specified number of sites of allelic imbalance (AI). The first group contained samples with ≦3 AI (N=10). The second group contained samples with <3 AI (N=21). The breast cancer-free survival interval, in months, is shown on the x-axis and the recurrence-free fraction is shown on the y-axis (p<0.018).

### DETAILED DESCRIPTION OF ILLUSTRATIVE EMBODIMENTS OF THE INVENTION

Most genomic DNA is identical between individuals of the same species. However, there are regions of DNA that can vary from individual to individual or cell to cell. Such variations in DNA sequence are termed “polymorphisms.” Short tandem repeats, also referred to herein as “STRs” or “microsatellite loci” are one class of DNA polymorphisms. STRs are short sequences of DNA, normally of two to five base pairs in length, which are repeated numerous times in a head-tail manner. The polymorphisms demonstrated by STRs are due to the different number of copies of the repeat element.

Because of their high degree of heterozygosity, STRs are widely used as genomic markers. As discussed in more detail in the “Short Tandem Repeat DNA Internet DataBase,” created by John M. Butler and Dennis J. Reeder of the Biotechnology Division, National Institute of Standards and Technology (NIST) (available on the worldwide web at cstl.nist.gov/div831/strbase), hundreds of STR loci have been mapped throughout the human genome. STR loci are found on almost every chromosome in the genome and several dozen are currently used in human identity testing (Hammond et al., Am. J. Hum. Genet. 1994, 55:175-189; Kimpton et al., PCR Meth. Appl. 1993, 3:13-22; Urquhart et al., Int. J. Leg. Med. 1994, 107:13-20; and Krenke et al, J Forensic Sci, 2002, 47:773-85). A variety of kits for the amplification of STR loci are commercially available, for example, from Promega (Madison, Wis.), Applied Biosystems (Foster City, Calif.), and Reliagene Technologies, Inc. (New Orleans, La.).

The present invention provides a simple, high throughput method for measuring the extent of allelic imbalance throughout a genomic nucleic acid sample. Allelic imbalance, also referred to herein as “AI,” results from the loss or gain of one of the two alleles at a genetic locus. The method of the present invention includes detecting short tandem repeat (STR) loci and calculating an allelic ratio for each STR loci. The STR loci are unlinked and located throughout the genome. In a preferred embodiment, STR loci are amplified, with the different alleles of each STR locus yielding separate amplicon products. The resultant amplicon products are then detected and quantified, and an allelic ratio is calculated for each STR loci.

To simplify the analysis, for a given STR locus, the allele with the greatest signal intensity is placed in the numerator, so that the resultant allelic ratio will always be 1.0 or greater. In normal cells, the ratio of two alleles' paired signal intensities following amplification would be expected to be about 1.0. Allelic imbalance at a given STR locus is present when the difference between the observed allelic ratio and 1.0 is statistically significant. Statistical significance may be determined by the investigator as appropriate for the specifics of the experimental data obtained. The determination of statistically significant allelic ratios is by methods known to the skilled artisan, using established techniques. Statistical tests including, for example, a one-way test, a two-way test, or the student's t test may be used to determine a statistically significant allelic ratio. Any of the methods of statistical analysis used in Example 1-5 may be used. Statistically significant allelic ratios can be used in the determination of allelic imbalance. For example, allelic ratios representative of allelic imbalance with about 90% statistical significance or greater, with about 95% statistical significance or greater, with about 97.5% statistical significance or greater, or with about 99% statistical significance or greater may be used to establish allelic imbalance. For example, allelic ratios of about 1.28 or greater, of about 1.37 or greater, of about 1.61 or greater, or about 2.15 or greater may be used as an indicator of allelic imbalance. In some embodiments, allelic ratios of about 1.28 or greater, of about 1.372 or greater, of about 1.61 or greater, or about 2.149 or greater may be used as an indicator of allelic imbalance. In some instances, an allelic ratio representing a minimum estimate of allelic imbalance may be used.

The method of the present invention may be used to determine allelic imbalance at one or more STR loci in a genomic nucleic acid sample. Any number of STR loci may be amplified. For example, one STR loci, two or more STR loci, three or more STR loci, four or more STR loci, five or more STR loci, ten or more STR loci, twelve or more STR loci, thirteen or more STR loci, fifteen or more STR loci, sixteen or more STR loci, twenty or more STR loci, twenty-one or more STR loci, thirty or more STR loci, thirty-two or more STR loci, and more STR loci may be amplified in the methods of the present invention. Further, at least 10 STR loci, at least 20 STR loci, at least 30 STR loci, at least 40 STR loci, at least 50 STR loci, at least 60 STR loci, at least 70 STR loci, at least 75 STR loci, at least 80 STR loci, at least 90 STR loci, at least 100 STR loci, at least 200 STR loci, or more STR loci may be amplified in the method of the present invention. When multiple STR loci are amplified, they are preferably unlinked and located throughout the genome. As used herein, unlinked STR loci are located on separate chromosomes or are widely separated on the same chromosome. Allelic ratios obtained may be the same or different for each of the various STRs amplified in a sample.

A wide variety of STR loci may be amplified in the methods of the present invention. STR loci to be amplified may include any of the STR loci discussed herein, any of the hundreds of known STRs, and newly identified STRs. For example, the STR loci to be amplified in the methods of the present invention may include one or more of amelogenin (chromosomal locations Xp22.1-22.3 and Yp11.2, see Shewale et al., “Anomalous Amplification of the Amelogenin Locus Typed by AmpFl STR® Profiler Plus™ Amplification Kit,” Forensic Science Communications, October 2000, Volume 2, number 4); CSF1PO (also known as CSF, chromosomal location 5q33.3-34 (human c-fms proto-oncogene for CSF-1 receptor gene), GenBank Accession No. X14720); D2S1338 (chromosomal location 2q35-37.1. GenBank Accession No. G08202); D3S1358 (chromosomal location 3p21, GenBank Accession No. 11449919); D5S818 (also known as D5, chromosomal location 5q21-q31, GenBank Accession No. G08446, Human Genome Database SequenceAC008512); D7S820 (also known as D7, chromosomal location 7q, GenBank Accession No. G08616, Human Genome Database Sequence AC004848); D8S1179 (also known as D6S502, chromosomal location 8q24.1-24.2, GenBank Accession No. G08710, Human Genome Database Sequence F216671); D13S317 (also known as D13, chromosomal location 13q22-q31, GenBank Accession No. G09017, Human Genome Database Sequence AL353628.2); D16S539 (also known as D16, chromosomal location 16q22-24, GenBank Accession No. G07925, Human Genome Database Sequence AC024591.3); D18S51 (chromosomal location 18q21.3, GenBank Accession No. X91254, Human Genome Database Sequence AP001534); D19S433 (chromosomal location 19q12-13.1, GenBank Accession No. G08036); D21S11 (chromosomal Location 21q21.1, GenBank Accession No. M84567, Human Genome Database Sequence AP000433); FGA (also known as FIBRA, chromosomal location 4q28 (located in the third intron of the human alpha fibrinogen gene), GenBank Accession No. M64982); TH01 (also known as HUMTH01 or TC11, chromosomal location 11p15-15.5 (located in intron 1 of the human tyrosine hydroxylase gene), GenBank Accession No. D00269); TPOX (also known as hTPO and TPO, chromosomal location 2p23-2pter (located in intron 10 of the human thyroid peroxidase gene), GenBank Accession No. M68651); or vWA (also known as VWF and VWA31A, chromosomal Location 12p12-pter, GenBank Accession No. M25858). All sixteen of amelogenin, CSF1PO, D2S1338, D3S1358, D5S818, D7S820, D8S1179, D13S317, D16S539, D18S51, D19S433, D21S11, FGA, TH01, TPOX, and vWA may be amplified in the present invention. One or more of these sixteen STR loci may be amplified along with one or more additional STR loci. Amelogenin, CSF1PO, D2S1338, D3S1358, D5S818, D7S820, D8S1179, D13S317, D16S539, D18S51, D19S433, D21S11, FGA, TH01, TPOX, and vWA represent sixteen unlinked, microsatellite loci located throughout the genome and are widely used as markers in the identification of human DNA. These sixteen STR loci are currently included in the commercially available AmpFISTR® multiplex PCR system (Applied Biosystems, Foster City, Calif.), described in more detail in the “AmpFISTR® Identifier T PCR Amplification Kit User's Manual” (Applied Biosystems 2001). While the STR loci referred to as amelogenin, also referred to as AMEL, is most often used to distinguish a male DNA sample from a female DNA sample, it may also be used in the present invention to demonstrate allelic imbalance in samples of male origin.

The STR loci to be amplified may include one or more of the thirteen core CODIS loci. CODIS is a National DNA Databank developed and maintained by the Federal Bureau of Investigation (FBI), for use in the identification of perpetrators of violent crime. In 1997, the FBI announced the selection of 13 STR loci to constitute the core of the CODIS national database. All CODIS STRs are tetrameric repeat sequences. The CODIS STR loci include D3S1358, vWA, FGA, D8S1179, D21S11, D18S51, D5S818, D13S317, D7S820, D16S539, TH01, TPOX, CSF1PO, and AMEL. One or more of D3S1358,vWA, FGA, D8S1179, D21S11, D18S51, D5S818, D13S317, D7S820, D16S539, TH01, TPOX, CSF1PO, or amelogenin may be amplified in the methods of the present invention. One or more of the CODIS loci may be amplified along with one or more additional STR loci. All thirteen CODIS loci may be amplified, with or without additional STR loci (Collins et al, J Forensic Sci, 2004, 49:1265-77).

The STR loci to be amplified in the methods of the present invention may include one or more of the various polymorphic DNA markers known to demonstrate a high frequency of loss of heterozygosity in breast cancer. See, for example, Moinfar et al., Cancer Res. 2000, 60:2562-6; O'Connell et al., J. Natl. Cancer Inst., 1998, 90:697-703; Kerangueven et al., Cancer Res., 1997, 57: 5469-5474; and Larson et al., Am J. Pathol. 2002, 161:283-90).

Desirable features for a STR loci to be amplified in the methods of the present invention may include one or more of the following; robust amplification under standard conditions, low amplification background, high heterozygosity, a regular repeat unit, distinguishable alleles, amplicon products that are easy to score and informative. With the present invention, STR loci may be amplified using any of a variety of DNA amplification procedures, to yield an amplicon product. For example, any of the various methods detailed in “DNA Amplification: Current Technologies and Applications,” Editors Vadim V. Demidov and Natalia E. Broude (Horizon Bioscience, Boston University, USA, 2004) may be used.

In some embodiments, a PCR-based technology may be used for DNA amplification. For example, a PCR based assay similar to that described by Skotheim et al. may be used for the amplification of STR loci (Skotheim et al., Cancer Genet Cytogenet. 2001, 127:64-70; and Sgueglia et al., Anal. Bioanal. Chem. 2003, 376:1247-54). PCR, a well-known and widely used technique in molecular biology, is a rapid, inexpensive and simple means of producing relatively large numbers of copies of DNA molecules from minute quantities of source DNA material, even when the source DNA is of relatively poor quality.

A primer is a short segment of nucleotides that is complementary to a section of the DNA that is to be amplified in a PCR reaction. A given STR may be amplified using PCR primers that bracket the locus. The length of the amplified DNA product, also referred to herein as an “amplicon,” will depend on the exact number of repeats at the STR locus. A wide variety of primers for amplifying STR loci are available. For example, any of the many primers described in Krenke et al., J. Forensic Sci. 2002, 47(4):773-785; Fregeau and Fourney, BioTechniques 1993, 15:100-119; Hammond et al., Am. J. Hum. Genet. 1994, 55:175-189; Kimpton et al., PCR Meth. Appl. 1993, 3:13-22; Urquhart et al., Int. J. Leg. Med. 1994, 107:13-20; Sprecher et al., BioTechniques 1996, 20:266-276; Urquhart et al., BioTechniques 1995, 18:116-121; Oldroyd et al., Electrophoresis 1995, 16:334-337; Lorente et al., Int. J. Leg. Med. 1993, 106:69-73; Roewer and Epplen, Forensic Sci. Int. 1992, 53:163-171; Sullivan et al., BioTechniques 1993, 15:637-641; Mannucci et al., Int. J. Leg. Med. 1994, 106:190-193; Nishimura and Murray, Nucleic Acids Res. 1992, 20:1167; Huang et al., Forensic Sci. Int. 1995, 71:131-136; or Masibay et al., J. Forensic Sci. 2000, 45(6):1360-1362 may be used.

By adjusting the distance of the primers from the STR repeat sequence, products can be obtained from different loci that do not overlap during gel electrophoresis. Primers are designed to bind specifically to the region of interest and not form primer dimers (that is, bind to themselves). Primers can be synthesized or obtained commercially, for example, from Research Genetics (Huntsville, Ala.), IDT (Coralville, Iowa), and Invitrogen (Carlsbad, Calif.). The amplification of multiple STR loci may be performed in several separate reaction mixtures or may be performed in a single reaction mixture. The coamplification of multiple STR loci in a single reaction mixture is referred to as a multiplex reaction or a multiplex PCR reaction (Kimpton et al., PCR Meth. Appl. 1993, 3:13-224; Kimpton et al., Int. J. Leg. Med. 1994, 106:302-311; and Edwards and Gibbs PCR Meth. Appl. 1994, 3:S65-S75).

In some embodiments, commercially available kits for the amplification of STR loci may be used. For example, the AmpFISTR® kit (Applied Biosystems, Foster City, Calif.) or the PowerePlex® system (Promega, Madison, Wis.) may used, following the manufacturer's instructions. The AmpFISTR® kit (Applied Biosystems, Foster City, Calif.) can be used, following the manufacturer's instructions, as detailed in the “AmpFISTR® Indentifier™ PCR Amplification Kit User's Manual” (Applied Biosystems 2001). The AmpFISTR® kit contains reagents that amplify 16 different STR loci within a single multiplex reaction.

After DNA amplification, the resultant STR amplicons are detected and quantified. The detection of the resultant STR amplicons may be carried out in a single preparation, or may be carried out in more than one or several preparations. Any of the various technologies available for detecting, resolving, or quantifying DNA products may be used, including, but now limited to, various electrophoretic and spectroscopic methods. For example, mass spectroscopy methods, including time-of-flight mass spectrometry (TOFMS), may be used in the analysis of STR amplicons. See Butler and Becker, “Improved Analysis of DNA Short Tandem Repeats with Time-of-Flight Mass Spectroscopy,” Science and Technology Research Report, U.S. Department of Justice, Office of Justice Programs, October 2001.

In preferred embodiments, any of the various electrophoretic separation technologies widely used in the analysis of nucleic acid products may be used to detect STR amplicons. For example, capillary electrophoresis may be used to resolve STR amplicons, one from another. Capillary electrophoresis (CE) encompasses a family of related separation techniques that use narrow-bore fused-silica capillaries to separate a complex array of large and small molecules. High electric field strengths are used to separate molecules based on differences in charge, size and hydrophobicity. Sample introduction can be accomplished by immersing the end of the capillary into a sample vial and applying pressure, vacuum or voltage. Automated capillary electrophoresis is available.

In some embodiments, to facilitate the detection of the resultant STR amplicons from one another, the PCR primers used may have fluorescent molecules covalently linked to the primer. To extend the number of different loci that can be analyzed in a single PCR reaction, multiple sets of primers with different “color” fluorescent labels may be used. Following the PCR reaction, internal DNA length standards are added to the reaction mixture and the DNAs are separated by length in a capillary gel electrophoresis machine. As DNA peaks elute from the gel they are detected with laser activation. The sequencing machines used for allele separation and detection are the same type currently being used in the Human Genome Sequencing project, with digital output that can be analyzed by special computer software.

The sample or samples on which the assays are performed may be obtained by any means. Samples may include cells, tissues and/or fluids. Samples may be obtained, for example, by needle-core biopsies, surgical biopsies, tissue excised during surgical procedures, and the like. A sample may be fresh, frozen, fixed in formalin or similar preservatives, embedded in paraffin, or preserved by similar tissue archival procedures. With tissues fixed in formalin or similar preservatives, the samples may be washed with an appropriate solution, such as phosphate buffered saline, to remove residual preservative prior to DNA purification. Fluid samples include, for example, blood, lymph, urine, cerebrospinal fluid (CSF) and nipple aspirate fluid (NAF). A sample may be obtained from a microbe, a plant, or an animal. An animal may include, for example, a rat, mouse, dog, cat, cow, horse, non-human primate, or human. A sample may be obtained from a model organism, including model systems used in the study of the mechanisms of allelic imbalance or studies of the mechanisms, diagnosis, or therapeutic treatment of cancer. A sample may be obtained by the in vitro cell culture of cells.

A sample may be obtained, for example, from a tumor, other cancerous tissues or cells, or precancerous tissues or cells. Cancers from which samples may be obtained include, but are not limited to, breast cancer, prostate cancer, renal cancer, and endometrial cancer. A sample may be obtained from histologically normal cells adjacent or proximate to a tumor. Such samples are also referred to herein as coexisting histologically normal (CHN) tissue. Such a sample may be obtained from a location at a distance away from the tumor, for example, about one centimeter distant from the tumor, about two centimeters distant from the tumor, about three centimeters distant from the tumor, about four centimeters distant from the tumor, about five centimeters distant from the tumor, about one to about five centimeters distant from the tumor, about seven centimeters distant from the tumor, or about ten centimeters distant from the tumor. A sample may be obtained in a patient from a site distal to a cancerous site, such as, for example, a site contralateral to the location of a tumor.

A sample may be obtained from inside the visible margin of a tumor. A sample may be obtained from outside, or distant from, the visible margin of a tumor. Such a sample may be obtained, for example, from about one centimeter outside the visible margin of the tumor, from about two centimeters outside the visible margin of the tumor, from about three centimeters outside the visible margin of the tumor, from about four centimeters outside the visible margin of the tumor, from about five centimeters outside the visible margin of the tumor, from about one to about five centimeters from outside the visible margin of the tumor, from about seven centimeters distant from outside the visible margin of the tumor, or from about ten centimeters outside the visible margin of the tumor.

A sample may be obtained from normal tissue, cells, or fluids. For example, a sample may be obtained from a normal subject, not undergoing a therapy or treatment. A sample may be obtained from normal tissues, cells, or fluids in a subject with cancer. A sample may be from a human patient undergoing diagnosis, treatment, or follow-up for cancer. “Treatment for cancer,” as used herein, includes therapies to decrease morbidity and mortality in a patient having cancer. Therapies include, for instance, chemotherapy and radiotherapy. A sample may be obtained as a part of the diagnosis of cancer. A sample may be obtained before a treatment is initiated, during a treatment, and/or after treatment has been completed. Samples may be taken from a given patient at one or more different times periods during the diagnosis, treatment, and/or follow-up for cancer. A sample may be obtained from an individual suspected of having a predisposition to cancer, for example, based on genetic or family history or exposure to environmental or behavioral risk factors.

A genomic nucleic acid sample may be extracted from the sample and purified by any conventional method. For example, tissue may be purified by means of a QIAamp™ tissue kit for isolation and purification of nucleic acids or a Qiagen DNAeasy Kit (both supplied by Qiagen, Valencia, Calif.) using the manufacturer's suggested protocols. Alternatively, either frozen, finely powdered tissue or suspensions of washed cells can be mixed with a lysis agent, such as 5 volumes of lysis buffer (0.1M EDTA, 0.5% Sarkosyl, pH 8.0) and 20 μg/μl boiled RNAase at 55° C. in a shaking water bath for 30 minutes. DNA can then be extracted, such as by addition of Proteinase K (United States Biochemical Corp., Cleveland, Ohio) to 200 μg/μl and after a suitable time, such as 4 hours, extracting the mixture, such as extraction twice with 2.5 volumes of a 1:1 mixture of phenol and chloroform, and twice with 2.5 volumes of chloroform alone. The solutions containing DNA can then by dialyzed and placed in a suitable buffer, such as dialyzed against TE buffer (1 mM EDTA, 10 mM Tris HCl, pH 7.8), precipitated with ethanol, resuspended in TE, and stored at 4° C. It is to be appreciated that the method employed to extract and purify genomic nucleic acid from a sample may be any method producing DNA of suitable purity that is compatible with the methods of analysis.

Very little nucleic acid sample is required in the methods of the present invention. An assessment of allelic imbalance can be obtained utilizing specimens with less than about 50 nanograms (ng) of genomic DNA, preferably with less than about 10 ng of genomic DNA, more preferably with less than about 5 ng of genomic DNA, and most preferably with less than about 1 ng of genomic DNA (the equivalent of approximately 150 cells).

The method described herein for the assessment of allelic imbalance has a number of significant advantages over existing technologies. This method is independent of the nature of the specimen. For example, the method can be used on samples that are frozen, formalin-fixed, or paraffin-embedded. The method does not require matched normal tissue, requires very little DNA, uses commercially available reagents, instrumentation and analysis software, can be applied to a variety of frozen and archival tissues, provides a quantitative basis for comparing the extent of allelic imbalance between samples, and provides information about both the fraction of genetically-altered cells in the population and the degree of heterogeneity in the genetically-altered fraction. The present invention provides a rapid, inexpensive and simple method of assessing allelic imbalance, in a format that is readily amenable to automated systems.

The present invention for the assessment of allelic imbalance may be used in the diagnosis of cancer. Using the methods described herein, one or more STR loci are amplified in a genomic nucleic sample suspected of containing cancerous cells or tissue and the extent of allelic imbalance assessed for each STR loci. The presence of allelic imbalance at one or more STR loci may be indicative of the presence of cancerous cells in the sample. In same instances, the presence of allelic imbalance at more than one STR loci is a more statistically significant indication that the sample contains cancerous cells. For example, the presence of allelic imbalance at two or more STR loci, three or more STR loci, four or more STR loci, five or more STR loci, or six or more STR loci indicates, with increasing statistical significance, the presence of cancerous cells in the sample. To assist in interpretation, an assessment of allelic imbalance from a sample suspected of containing cancerous cells may be compared assessments of allelic imbalance obtained from references tissues obtained from normal, non-cancerous tissues. Such comparisons may also be made to normalized or average patient populations, with adjustment for age, sex, race, or other factors, as appropriate.

The present invention for the assessment of allelic imbalance may be used in the identification of the margin of a tumor. Using the methods described herein, one or more STR loci are amplified in a genomic nucleic sample obtained from a tissue sample from a location adjacent to a tumor and the extent of allelic imbalance assessed for each STR loci. The presence of allelic imbalance at one or more STR loci is indicative of the presence of cancerous cells and/or precancerous cells in the sample. In some instances, the presence of allelic imbalance at more than one STR loci is a more statistically significant indication that the sample contains cancerous cells. For example, the presence of allelic imbalance at two or more STR loci, three or more STR loci, four or more STR loci, five or more STR loci, or six or more STR loci indicates, with increasing statistical significance, the presence of cancerous cells in the sample. Such a determination that a sample obtained from tissue adjacent to a tumor contains cancerous or precancerous cells indicates that the sample is to be considered as being within the margin of the tumor. Such a method for determining the margins of a tumor may be useful for determining tissue boundaries in surgical procedures to resect cancerous or precancerous tissues in a patient. Such a method is useful for the identification of abnormal cells that are not identifiable by conventional pathological evaluation techniques and for the identification of cells populations that are pre-cancerous.

Currently available prognostic markers often fail to identify patients with lethal, metastatic tumors. Accordingly, cancer patients often receive empiric, aggressive therapies that, in fact, may not be necessary. Thus, there is a pressing need to identify more informative prognostic markers. With the present invention it has been discovered that tumors with higher levels of allelic imbalance have more aggressive phenotypes than tumors with lower levels of allelic imbalance. Thus, the methods of the present invention, for the assessment of allelic imbalance, have significant prognostic value and may be employed for the determination of treatment plans, treatment options, and the like. Further, the present invention demonstrates that an assessment of the extent of allelic imbalance in coexisting histologically normal tissues is of independent prognostic significance.

Cells containing more extensive genomic alterations have the greatest probability of acquiring tumor-promoting phenotypes, such as extended life span, extravasation, angiogenesis, and metastasis. An assessment of the extent of allelic imbalance can serve as a surrogate for chromosomal instability and, consequently, can provide an assessment related to prognosis in cancer. An increase in the extent of allelic imbalance can be associated with an increased risk of metastasis, an increased risk of recurrence and/or a reduced overall survival rate.

The present invention for the assessment of allelic imbalance may be used for determining the prognosis of a patient with cancer. Using the methods described herein, one or more STR loci are amplified in a genomic nucleic sample obtained from the patient and the extent of allelic imbalance assessed for each STR loci. A determination of allelic imbalance at an increasing number of STR loci may indicate an increased likelihood that the cancer is an aggressive cancer, with an increased risk of metastasis, recurrence and/or death. To assist in interpretation, the assessment of allelic imbalance from the cancerous sample may be compared to allelic imbalance assessments obtained from various patient populations, with known cancer outcomes.

The invention disclosed herein further demonstrates that an increased extent of allelic imbalance in the coexisting histologically normal tissues adjacent to a tumor also has prognostic value and can predict disease recurrence. Thus, the present invention may be used for determining the prognosis of a patient with cancer, by obtaining a sample of coexisting histologically normal (CHN) tissue from the patient and assessing the extent of allelic imbalance in the coexisting histologically normal tissue sample. Such a coexisting histologically normal tissue sample may be obtained, for example, from a site at least about one centimeter distal from any histologically abnormal tissue, from a site at least about one centimeter to about five centimeters distal from any histologically abnormal tissue, from a site within about five centimeters of histologically abnormal tissue, or from a site at least about five centimeters distal from any histologically abnormal tissue. One or more STR loci are amplified in a genomic nucleic acid sample from the CHN sample and the extent of allelic imbalance assessed for each STR loci. A determination of allelic imbalance at an increasing number of STR loci may indicate an increased likelihood that the cancer is an aggressive cancer, with an increased risk of metastasis, recurrence and/or death. To assist in interpretation, the assessment of allelic imbalance from the CHN sample may be compared to allelic imbalance assessments obtained in various patient populations, as appropriate. The discovery of the present invention, that an increased extent of allelic imbalance in coexisting histologically normal tissues is associated with disease recurrence and survival has several important implications. For example, coexisting histologically normal cells that are truly normal and contaminate a tumor specimen will not diminish the prognostic value of the assay, thus precluding the necessity of microdissection. Further, the genetic events that influence a tumor's potential to produce aggressive disease may occur early in tumorigenesis, or independent of tumorigenesis, prior to phenotypic changes.

The methods of the present invention may be used to monitor the responsiveness of a cancer to therapy. Samples can be taken from a patient prior to the commencement of a cancer treatment, at various intervals during cancer treatment, and/or after the completion of cancer treatment. For each sample, an assessment of the extent of allelic imbalance can be determined, using the methods described herein. A decrease in the number of STR loci exhibiting allelic imbalance in samples taken during and after cancer treatment, when compared to the number of STR loci exhibiting allelic imbalance prior to the commencement of therapy is indicative that the number of cancerous cells in the sample has decreased and that the cancer is responsive to therapy.

The methods of the present invention may be used in predicting responsiveness to a cancer therapy. Samples can be taken from a patient prior to the commencement of a cancer treatment and the extent of allelic imbalance in the sample determined, using the methods described herein. For a given cancer, the results obtained can be compared to allelic imbalance assessments from samples obtained from earlier patients with similar cancers.

The methods of the present invention may also be used to identifying a predisposition to cancer. Samples can be taken from an individual with a suspected predisposition to cancer and the extent of allelic imbalance in the sample determined, using the methods described herein. An allelic imbalance in at least one STR locus may indicate that the subject has a predisposition to cancer.

The methods of the present invention may also be used in conjunction with various methods of assessing telomere DNA content; including, for example, the methods discussed in U.S. Patent Application 20040234961 and U.S. Pat. Nos. 5,489,508; 5,695,932; 5,834,193; 5,871,926; 6,235,468; and 6,297,356.

The assays and methods described herein may also be used for monitoring of progression of diseases other than cancer, and further for the determination of the efficacy of therapies in addition to cancer therapies.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Measurement of Genome-Wise Allelic Imbalance in Human Tissue Using a Multiplex PCR System

The present example describes a method for measuring the extent of allelic imbalance throughout the genome.

Materials and Methods

Tissue Acquisition. Buccal cells were collected from the oral rinses of randomly selected volunteers, independent of gender or age. Frozen renal tissues, including both renal cell carcinomas and, in most instances, matched normal tissue, were obtained from radical nephrectomies and provided by the Cooperative Human Tissue Network. Two independent sets of archival invasive breast tumors, one comprised of frozen tissues and another of formalin-fixed, paraffin-embedded tissues, both obtained from lumpectomies or radical mastectomies, were provided by the University of New Mexico Solid Tumor Facility and New Mexico Tumor Registry Tissue Acquisition Service (NMTR-TAS), respectively. Formalin-fixed, paraffin-embedded archival prostate tissues, obtained from radical prostatectomies, were also provided by the NMTR-TAS. All specimens lacked patient identifiers and were obtained in accordance with all federal guidelines as approved by the University of New Mexico Human Research Review Committee.

DNA Isolation and Quantification. DNA was isolated from all tissue samples using the DNeasy® silica based spin column extraction kits (Qiagen; Valencia, Calif.), and the manufacturer's suggested protocol for animal tissues. Paraffin embedded samples were treated with xylene to remove paraffin prior to DNA extraction, and further purified using silica spin filters (MO BIO; Carlsbad, Calif.). DNA was quantified with Picogreen® fluorescent-based quantification reagent kit (Molecular Probes; Eugene, Oreg.).

Multiplex Polymerase Chain Reaction (PCR) Amplification of STR Loci. The AmpFISTR® kit (Applied Biosystems, Foster City, Calif.) was used to amplify 16 different short tandem repeat (STR) microsatellite loci (Amelogenin, CSF1PO, D2S1338, D3S1358, D5S818, D7S820, D8S1179, D13S317, D16S539, D18S51, D19S433, D21S11, FGA, TH01, TPOX and vWA) in a single multiplexed PCR reaction, according to the supplier's protocol. The 16 primer sets are designed and labeled to permit the discrimination of all amplicons in a single electrophoretic separation. The PCR products were resolved by capillary electrophoresis using an ABI Prism 377 DNA Sequencer (Applied Biosystems, Foster City, Calif.). Fluorescent peak heights were quantified using ABI Prism GeneScan Analysis software (Applied Biosystems, Foster City, Calif.) and statistical analyses were performed using SAS JMP® software (version 5.1).

Results and Discussion

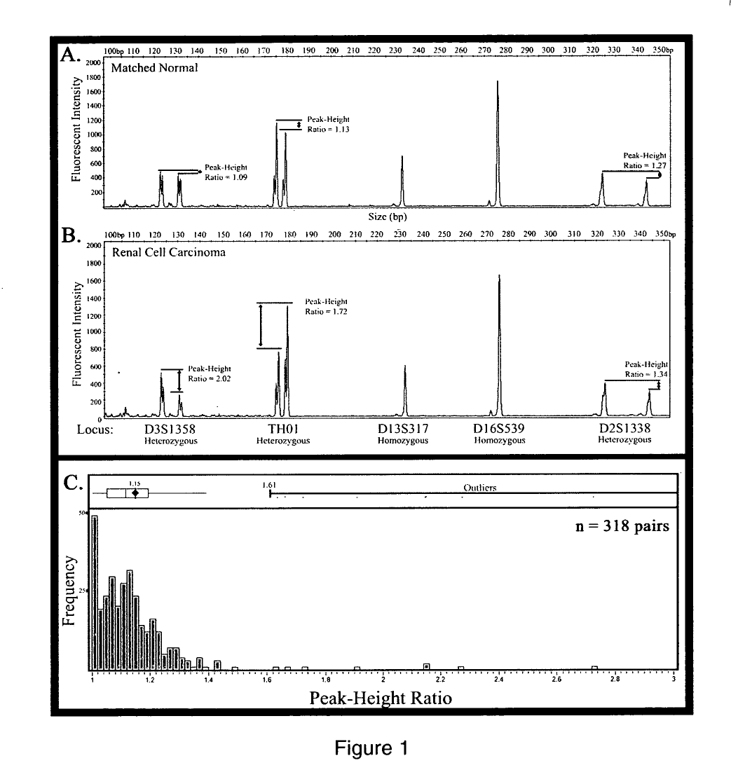

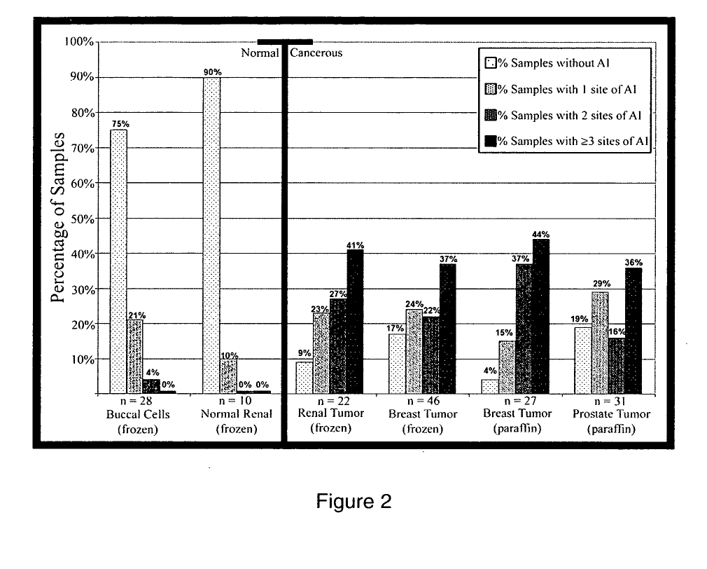

The 16 allelic microsatellite loci amplified by the AmpFISTR® primer set are unlinked, and can be used to evaluate allelic imbalance at several arbitrary sites throughout the genome. During the PCR reaction, each amplicon is labeled with one of four fluorescent dyes (6-FAM, PET, VIC and NED), each with a unique emission profile, thus allowing the resolution of amplicons of similar size. FIG. 1A (upper panel) shows the separation of the subset of VIC-labeled amplicons derived from normal renal tissue. Two of the allelic pairs are homozygous, as indicated by a single peak, and three of the allelic pairs are heterozygous, as indicated by two peaks. Although the peak heights varied between loci, the peak heights of the paired alleles were similar. Ideally, the ratio of two alleles following PCR amplification would be 1.0 in normal tissues. To test this premise, the ratios of paired alleles' signal intensities were compared at 318 heterozygous loci in buccal cells from 28 healthy individuals (FIG. 1C). To simplify the analysis, the allele with the greater fluorescence was always made the numerator, so the ratio was always greater than or equal to 1.0. As expected, the median and mean ratios were near 1.0 (median=1.11, mean=1.15, SD 0.18). Any allelic ratio greater than the value of an outlier (i.e. 1.61, 2.2% of total), was defined as abnormal. Using this standard, 120 heterozygous loci in 10 independent samples of normal renal tissue were evaluated (FIG. 2). An allelic ratio greater than 1.61 was detected at only one locus (0.83%).

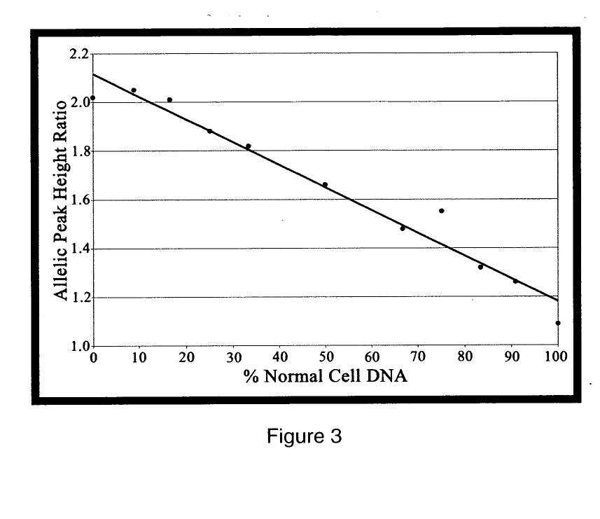

Allelic imbalance (AI) results from the loss or gain of one of the two alleles in all, or a subset of cells in a tumor. Therefore, the ratio of paired alleles' signal intensities is expected to be greater than 1.0 at each locus with allelic imbalance, with the observed ratio primarily dependent on the fraction of cells in the sample with allelic imbalance. This is shown in FIG. 1B (lower panel) where the peak heights were greater than 1.61 at two of the three heterozygous loci in the tumor tissue from the same patient. Using this approach, the frequency of allelic ratios greater than 1.61 at 1450 heterozygous loci in DNA purified from 126 frozen or paraffin-embedded renal, breast and prostate tumors was determined (FIG. 3). In contrast to normal cells, allelic imbalance was detected at 268/1450 loci (18.5%), 10 times greater than the frequency in the normal tissues.

Seven (18%), 1 (2.6%), and 0 (0%) of the 38 buccal and renal samples contained one, two and three loci with allelic imbalance, respectively. Based on these data, less than 1% of tumor samples would be expected to contain three or more loci with allelic imbalance (i.e. 0.18×0.18×0.18). However, at least 35% of the tumors contained three or more loci with allelic imbalance, independent of their site of origin or methods of preservation (FIG. 3). The data in FIG. 2 is a minimum estimate of allelic imbalance, since the assay cannot discriminate between homozygous alleles and complete loss of heterozygosity in the absence of matched normal tissue. This limitation is mitigated by the near ubiquitous presence of normal tissue within tumors. For example, an allelic ratio of 1.61, which is used as the definition for allelic imbalance in this example, could represent either a population comprised of 60% cells heterozygous for the allele and 40% with complete loss of one allele, or 40% cells heterozygous for the allele and 60% with duplication of one allele.

In order to assess the affects of differing cellular compositions on the observed allelic ratios, defined mixtures of DNAs were constructed from the paired normal and cancerous renal tissue shown in FIG. 1. As shown in FIG. 3 for the D3S1358 locus, there was a linear relationship (R=0.96) between the ratio of alleles and the composition of the mixture. Similar results were obtained for each of the other loci exhibiting allelic imbalance. These results indicate that the allelic ratios also provide information on the fraction of normal cells in the tumor sample.

It is also well established that tumor cells are genetically heterogeneous. Therefore, one would expect that the allelic ratios between specific loci would differ, reflecting this heterogeneity. As a representative example, the heterozygous allelic peak height ratios from two tumors are provided. For tumor 2064 the heterozygous allelic peak height ratios were 1.01, 1.01, 1.15, 1.29, 1.32, 1.33, 1.33, 1.36, 1.37, 1.39, 1.90, 1.95, and 2.25 (with peak height ratios indicating allelic imbalance indicated by bold). For tumor 1855 the heterozygous allelic peak height ratios were 1.01, 1.04, 1.06, 1.07, 1.12, 1.15, 1.16, 1.23, 1.87, 1.88, 2.01, 3.78, and 3.84 (again, peak height ratios indicating allelic imbalance are indicated by bold). In tumor 2064, the allelic ratios are approximately 2.0 at all three loci with allelic imbalance (D3S1358, TH01, D 18S51), implying that most tumor cells are genetically similar. In contrast, in tumor 1855, the allelic ratios are approximately 1.9 at three of five loci with allelic imbalance (D3S1358, TPOX, VWA) and 3.8 at the remaining two loci with allelic imbalance (D8S1179, D2S1338). This implies that tumor 1855 contains at least two genetically distinct tumor cell populations, all of which have allelic imbalance at the D8S1179 and D2S1338 loci, and only some of which have allelic imbalance at the D3S1358, TPOX and VWA loci.

This example has described a simple method for measuring the extent of allelic imbalance throughout the genome. This method has a number of significant advantages over existing technologies. Matched normal tissue is not required. The method utilizes commercially available reagents, instrumentation, and analysis software. The method can be applied to a variety of fresh, frozen and archival tissues. Very little DNA is required (the equivalent of approximately 150 cells). The method provides a quantitative basis for comparing the extent of allelic imbalance between samples. And, the ratios of the alleles provide information about both the fraction of genetically altered cells in the population and the degree of heterogeneity in the genetically altered fraction.

### Example 2

### Telomere DNA Content in Prostate Tumors and Coexisting Histologically Normal Tissues is Associated with Allelic Imbalance and Disease-Free Survival

In this example, PCR was used to detect allelic imbalance (AI) at sixteen microsatellite loci in archival tumor (N=31) and paired coexisting histologically normal (CHN) prostate tissues (N=27). Slot blot assay was used to quantitate telomere DNA content (TC) in archival tumor (N=77) and CHN prostate tissues (N=53). Cox proportional hazards analysis related TC, age at diagnosis, Gleason sum score and pelvic node involvement to the time of prostate cancer recurrence. The incidence of allelic imbalance in tumor and CHN tissues was identical. TC was not associated with the fraction of normal tissue within the tumor specimen. TC was associated with the number of sites of allelic imbalance in tumors (p=0.032) and CHN tissue (p=0.037). TC in tumors and CHN tissues from men whose cancers recurred were each lower than TC in prostate tumors from the men whose cancer did not recur within six years (p=0.0123, p=0.0244, respectively). TC was a predictor of time to prostate cancer recurrence controlling for age, Gleason sum score, and pelvic node involvement (RH=5.02, 95% CI 1.40-17.96, p=0.0132). This example demonstrates that dysfunctional telomeres occur in both prostate tumors and CHN tissue, implying genomic instability may be characteristic of the environment in which a prostate tumor develops, rather than of the tumor itself. TC was associated with cancer-free interval, further implying TC could be an independent prognostic marker in prostate cancer.

Materials and Methods

Study Group. The study group was comprised of randomly selected men diagnosed with prostate cancer who were treated with radical prostatectomy at the University of New Mexico Hospital between 1982 and 1995. Paraffin embedded prostate tumor tissue, coexisting histologically normal (CHN) prostate tissue from outside the tumor margin, and associated patient data were obtained by the New Mexico Tumor Registry in accord with all federal regulations, as approved by the University of New Mexico Health Science Center Human Research Review Committee. Patient data included age at diagnosis, pelvic lymph node involvement, Gleason sum score and prostate cancer recurrence. Prostate cancer recurrence was defined as documented distant metastasis, biochemical recurrence (rising PSA) or death as a consequence of prostate cancer. Subjects did not receive additional treatments prior to disease recurrence. Buccal cells were obtained from anonymous, healthy volunteers without regard to age or gender.

Histological Review. Serial sections, 25 micrometer (μm)-thick, were cut from paraffin blocks containing tumor or CHN tissues. Representative sections of tumor and CHN tissues were stained with hematoxylin and eosin and examined microscopically. Histopathological assessment of tumor and CHN tissue was confirmed and the fractions of the field containing normal and tumor cells were determined.

Determination of Allelic Imbalance (AI). DNA was extracted from twelve, 25 μl-thick sections of paraffin-embedded tissue or from frozen buccal cell pellets and quantified, each as described previously (Stewart et al., Proc. Natl. Acad. Sci. USA, 2002, 99:12606; Zhu et al., Proc. Natl. Acad. Sci. USA, 1999, 96:3723; Artandi et al., Curr Opin Genet Dev, 2000, 10:39-46; Karan et al., Int J Cancer 2003, 103:285-93; Zitzelsberger et al., Br J Cancer 2001, 84:202-8; Donaldson et al., J. Journal of Urology, 1999, 162:1788-92; and Fordyce et al., Biotechniques 2002, 33:144-8). Allelic imbalance was evaluated using a AmpFIISTR® kit (Applied Biosystems, Foster City, Calif.), amplifying 16 different short tandem repeat (i.e. microsatellite) loci within a single multiplex reaction. The amplicons from this reaction are separated by capillary electrophoresis and histograms of the fluorescently labeled products are generated. Approximately 1 nanogram (ng) of DNA was amplified in a standard 25 microliter (μl) reaction mix according to the manufacturer's protocol. In each reaction there was 10 μl of the reaction mix, 5 μl of the “Identifier” Primer Set and 2.5 units (U) of AmpliTaq Gold DNA polymerase. Cycling conditions included an initial denaturation at 95° C. for 11 minutes followed by 30 cycles of 1 minute at 94° C., 1 minute at 59° C., and 1 minute at 72° C., with a final extension of 60 minutes at 60° C. PCR products were resolved by capillary electrophoresis and detected using an ABI Prism 377 DNA Sequencer (Perkin Elmer, Foster City, Calif.). Data was analyzed by the ABI Prism GeneScan and Genotyper Analysis software (Applied Biosystems, Foster City, Calif.). Since matched normal tissue from a distant site was not available for these samples, allelic imbalance was defined by the ratio of fluorescence in each histogram peak derived from heterozygous alleles. Ideally, the ratio would be 1.0 in normal tissues, and greater or less than 1.0 in tissues with allelic imbalance. To simplify the analysis, the allele with the greater fluorescence was always the numerator, so the ratio was always greater than 1.0. The observed ratio is dependent on both experimental variables (e.g. the amplification efficiencies of the two alleles) and the fraction of cells in the sample with allelic imbalance. The sixteen pairs of unlinked allelic loci detected by the primer set are not associated with frequent sites of loss of heterozygosity in prostate tumors and, therefore, provide an unbiased means to assess the degree of instability throughout the genome. The assay was calibrated by measuring peak ratios at 318 heterozygous loci in buccal cell DNA obtained from 28 anonymous, healthy volunteers without regard to age or gender (Table 1). The mean peak-height ratio was 1.14 (SD 0.18). The cutoff point for an outlier is defined as [3×Interquartile Range+the 75th percentile] and was determined to be a peak-height ratio above 1.61. Hence, any heterozygous allele-pair with a peak-height ratio greater than 1.61 was classified as allelic imbalance (AI).

Determination of Telomere DNA Content (TC). Slot blots were prepared and analyzed as described (Van Steensel et al., Cell 1998, 92:401-413). The reproducibility of each experiment was verified by comparing the TCs of HeLa and placenta DNA analyzed on the same blot. In most instances, DNA from each tumor tissue was analyzed independently three times, each in triplicate. The coefficient of variation was less than 10%.

Statistical Methods. Nonparametric Wilcoxon/Kruskal-Wallis analysis was used to assess the significance of the relationships between TC, the number of sites of allelic imbalance, and prostate cancer recurrence. Cox proportional hazards analysis was used to compute the risk for prostate cancer recurrence associated with TC, Gleason sum score and pelvic node involvement.

TABLE 1
Allelic Imbalance in Normal Buccal Cells, Prostate Tumors and
Coexisting, Histologically Normal (CHN) Prostate Tissues.

| | Normal Buccal | Prostate | Prostate |
|---|---|---|---|
| | Cells | Tumors | CHN¹Tissue |
| # Samples | 28 | 31 | 27 |
| # Heterozygous Loci | 318 | 411 | 342 |
| # Samples with 0 sites | 21 (75%) | 6 (19%) | 5 (19%) |
| AI² | | | |
| # Samples with 1 site | 6 (21%) | 9 (29%) | 7 (26%) |
| AI | | | |
| # Samples with 2 sites | 1 (4.0%) | 5 (16%) | 5 (19%) |
| AI | | | |
| # Samples with ≧3 | 0 | 11 (35%) | 10 (37%) |
| sites AI | | | |

Notes:
¹CHN: Coexisting, Histologically Normal (CHN) Prostate Tissues.
²AI: Allelic imbalance.

Results

Reduced Telomere DNA Content in Prostate Tumors are Associated with Allelic Imbalance. To establish that critically shortened, dysfunctional telomeres generate genomic instability and thus, phenotypic variability in neoplastic prostate tissues, a multiplex PCR-based method for assessing allelic imbalance (AI) at sixteen pairs of unlinked microsatellite loci was employed. Allelic imbalance was detected in approximately 2.5% (8/318) of the loci in normal buccal cells. Six (21%), 1 (3.6%), and 0 (0%) of the 28 samples contained one, two and three or more sites of AI, respectively (Table 1). The numbers of sites of AI at 411 heterozygous loci in 32 prostate tumors was also measured. Approximately 35% of the tumors contained three or more sites of AI (Table 1), consistent with the accepted view that amplification, loss or structural rearrangement of chromosomal domains occurs in virtually all cancers, including prostate cancer (Karan et al., Int J Cancer 2003, 103:285-93; and Zitzelsberger et al., Br J Cancer 2001, 84:202-8).

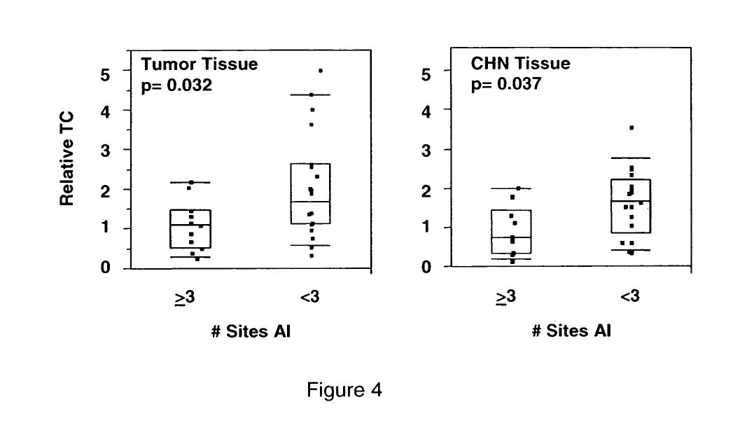

Chromosome breakage-fusion cycles that result from dysfunctional telomeres are a potential cause of allelic imbalance. If dysfunctional telomeres resulting from telomere attrition are a significant factor in the genesis of allelic imbalance, then telomere length would be expected to be associated with the extent of allelic imbalance. To test this prediction, the content of telomere DNA (TC), a surrogate for telomere length, was measured in the same DNA samples and compared to the number of loci with allelic imbalance. As shown in FIG. 4, there was a clear difference in the distribution of TC values in tumors with 0-2 sites of allelic imbalance compared to those with 3 or more sites of allelic imbalance. Non-parametric Wilcoxon/Kruskal-Wallis Rank Sums Test revealed a significant association between TC in prostate tumor tissues and the number of sites of allelic imbalance (p=0.032).

Telomere Dysfunction in Histologically Normal Prostate Tissue. Histological analysis of representative tumor sections revealed variable contents of normal prostate tissue coexisting with the tumor. The fraction of normal tissue (defined by area) ranged from 0-80% of the fields (mean, median each 40%). The sensitivity of the assay to detect allelic imbalance is dependent on the cellular heterogeneity of the sample. Since normal cells typically have few sites of allelic imbalance (Table 1), the potentially confounding effects of the contaminating normal cells on the determination of allelic imbalance in the tumor were a concern. Therefore, allelic imbalance and TC were measured in paired specimens of tumor-free, coexisting histologically normal (CHN) prostate tissue obtained from sites outside the tumors' margins. All samples came from independent paraffin blocks. The percentage distribution of the number of sites of allelic imbalance in prostatic CHN tissues was virtually identical to that measured in prostate tumor tissues (Table 1). Similarly, non-parametric Wilcoxon/Kruskal-Wallis Rank Sums Test revealed a significant association between TC in CHN prostate tissues and the number of sites of allelic imbalance (p=0.037).

Eighteen of the 27 CHN samples were from the same patients as the tumor tissue. The 18 pairs of patient-matched tumor and CHN tissues contained 229 heterozygous loci. Allelic imbalance was detected at 25 and 27 loci in the tumors and CHN tissues, respectively, 10 of which were common to both samples. This does not appear to be the result of selection of “hot spots” of allelic imbalance, since 7 of the 10 loci are unique and located on different chromosomes. The chances of this result occurring by chance are approximately 0.001%. These data strongly suggest a commonality between the nature and extent of genomic instability in the tumor and CHN tissues.

Telomere DNA Content in Prostate Tumors and CHN Tissues Correlate with Disease Recurrence. Prior studies have demonstrated significant associations between TC and recurrence and survival in a case control study of prostate cancer (Donaldson et al., J. Journal of Urology, 1999, 162:1788-92) and between TC and aneuploidy and nodal involvement in women with breast cancer (Griffith et al., Breast Cancer Research & Treatment, 1999, 54:59-64). To establish that genomic instability resulting from dysfunctional telomeres generates phenotypic variability that, in turn, promotes the genesis of lethal, metastatic tumor cells, a retrospective study was performed of the relationship between TC in archival prostate tumors and prostate cancer-free interval in a cohort of men treated with prostatectomy between 1982 and 1995.

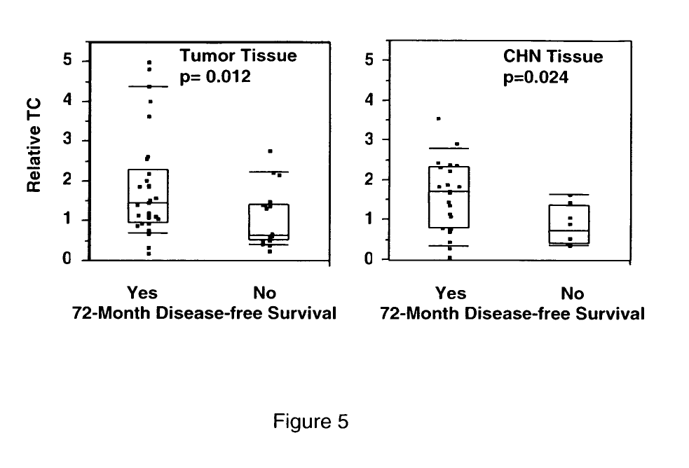

As shown in Table 2, most tumors were Gleason Grade of 6-7 and had not spread to the pelvic nodes. Nineteen of these men developed recurrent prostate cancer, 5 died from causes unrelated to prostate cancer and 53 men remained free of recurrent prostate cancer, 30 of whom were free of prostate cancer for at least six years. As shown in FIG. 5, the TC in prostate tumors from men that subsequently developed recurrent disease was lower than TC in prostate tumors from the men that did not recur within 6 years (p=0.0123). Likewise, the TC in CHN prostate tissue from men that subsequently developed recurrent disease was lower than TC in CHN tissue in the subgroup of men that did not recur within 6 years (p=0.0244).

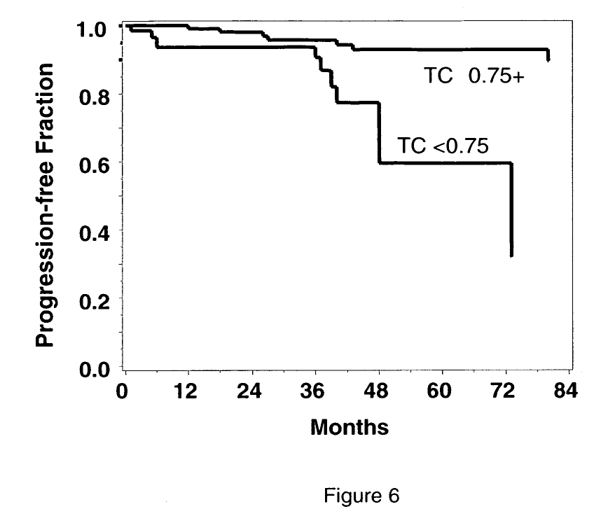

Telomere DNA Content in Prostate Tumors Correlates with Cancer-free Interval. The cohort was divided into three groups based on TC (<0.75, 0.75-1.49, >1.5) and a Cox proportional hazards model of time until recurrence or death from prostate cancer was developed (Table 3). The variables included tumor TC, age at diagnosis, pelvic node involvement and Gleason sum score. There was no increased risk of recurrence associated with TC values of 0.75 to 1.49. However, TC values <0.75 conferred a relative hazard of 5.02 (p=0.013). By comparison, the hazard conferred by pelvic node involvement and Gleason sum scores of eight or more were 6.50 (p=0.0002) and 5.96 (p=0.021), respectively). Recurrence-free survival for men with TC of 0.75 and above and less than 0.75 is shown in FIG. 6.

TABLE 2
Characteristics of Prostate Cohort

| | | Months | Age at | Gleason | Pelvic Nodes |
|---|---|---|---|---|---|
| | N | Follow Up¹ | Diagnosis | Sum Score | Yes/No/Unknown |
| Recurrence² | | | | | |
| N | 19 | | | | 7/11/1 |
| Range | | 6-143 | 52-72 | 3-9 | |
| Mean (SD) | | 63 (38.5) | 64 (5.4) | 7 (1.5) | |
| Median | | 48 | 65 | 7 | |
| No Recurrence | | | | | |
| Subgroup 1 | | | | | |
| Follow Up ≧ 72 Months | | | | | |
| N | 30 | | | | 0/29/1 |
| Range | | 72-175 | 54-76 | 1-9 | |
| Mean (SD) | | 98 (24.4) | 67 (5.5) | 6 (1.7) | |
| Median | | 93 | 67 | 7 | |
| Subgroup 2 | | | | | |
| Follow Up < 71 Months | | | | | |
| N | 23 | | | | 3/16/4 |
| Range | | 33-70 | 52-73 | 3-9 | |
| Mean (SD) | | 56 (8.9) | 66 (6.8) | 6.6 (1.5) | |
| Median | | 58 | 66 | 7 | |
| Subgroup 3 | | | | | |
| Death Not by Cancer | | | | | |
| N | 5 | | | | 0/5/0 |
| Range | | 67-118 | 63-71 | 1-8 | |
| Mean (SD) | | 92 (18.7) | 67 (3.4) | 5 (2.5) | |
| Median | | 96 | 68 | 5 | |

Notes:
¹Months of follow up after prostatectomy.
²Documented distant metastasis, biochemical recurrence (rising PSA) or death as a consequence of prostate cancer within 72 months after prostatectomy.

TABLE 3
Progression Free Survival by Telomere DNA Content, Adjusted for
Age, Gleason Score, and Pelvic Node Involvement.

| Variable | Level | RH¹ (95% CI) | p-Value |
|---|---|---|---|
| Age | Slope (per 10 years) | 0.28 (0.10, 0.77) | 0.0133 |
| Gleason score | 2-6 | 1.00 | |
| | 7 | 4.54 (1.17, 17.72) | 0.0292 |
| | 8-9 | 5.96 (1.31, 27.17) | 0.0210 |
| Pelvic Nodes | Negative | 1.00 | |
| | Positive | 6.50 (2.41, 17.51) | 0.0002 |
| TC² | ≧1.50 | 1.00 | |
| | 0.75-1.49 | 1.00 (0.22, 4.65) | 0.9992 |
| | <0.75 | 5.02 (1.40, 17.96) | 0.0132 |

Notes:
¹Relative Hazard (RH) and 95% Confidence Intervals (CI) from Cox Proportional Hazards Model of Time Until Recurrence or Death from Prostate Cancer.
²Telomere DNA content (TC).

Discussion

At least three principal conclusions are obtained from the present study. A first conclusion is that the number of sites of allelic imbalance in prostate tumors is correlated with the content of telomere DNA in the tumor. It has been hypothesized that critically shortened, dysfunctional telomeres generate genomic instability in neoplastic prostate tissues, including truncation, deletion and amplification of chromosomal loci (Gisselsson et al., Proc. Natl Acad. Sci USA, 2001, 98:12683-12688; Hackett et al., Cell, 2001, 106:275-286; Lo et al., Neoplasia, 2002, 4:531-538; Lundblad, Current Biology, 2001, 11:R957-960; and O'Hagan et al., Cancer Cell, 2002, 2:149). Although there are several potential causes of allelic imbalance, the strong association between TC and the number of sites of allelic imbalance is consistent with the conclusion that telomere dysfunction is a significant cause of genomic instability in human prostate tumors. Similarly, O'Sullivan and colleagues have recently reported that chromosomal instability in ulcerative colitis is due to telomere shortening (O'Sullivan et al., Nat. Genet. 2002, 32:280-284).

A second conclusion from the present example is the highly significant and unexpected conclusion that allelic imbalance and telomere DNA content in histologically normal (CHN) prostate tissue outside the tumor margin are quantitatively similar to allelic imbalance and telomere DNA content in the tumor itself. This conclusion is founded on the virtual identical frequencies of allelic imbalance in tumors and CHN tissue, the conservation of specific sites allelic imbalance in tumors and CHN tissues, the similar relationship between TC and allelic imbalance in tumors and CHN tissues, and the relationship between TC in tumors and CHN tissue and prostate cancer recurrence. Together, these data imply that TC, and resulting allelic imbalance, are characteristics of the cellular environment in which a prostate tumor develops, rather than characteristics of the tumor itself. Thus, genetic events that influence a tumor's potential to produce metastatic disease may occur early in tumorigenesis, prior to phenotypic changes, or even be independent of tumorigenesis. In this context, inherited or environmental conditions that affect TC in the prostate could be risk factors for tumor progression.

The mechanisms that lead to telomere attrition and dysfunction in CHN tissues are not known. Although prostate tumor cells typically have shorter telomeres than somatic cells (Meeker et al., Cancer Res. 2002, 62:6405), a presumed consequence of increased proliferation, it is difficult to explain why telomeres also would be altered in CHN tissues distal to the tumors' margins. However, Vukovic and colleagues have similarly reported that TC is reduced in high grade prostatic intraepithelial neoplasia (PIN) proximal to prostate tumors, and greater in high grade PIN distal to the tumor (Vukovic et al., Oncogene 2003, 22:1978). Since CHN tissue was obtained from separate blocks with no apparent tumor cell involvement, the tissue domains containing the reduced TC appear to be extensive and, thus, could reflect local differences in all or part of the prostate gland.

A third important conclusion from this example is that TC in prostate tumor DNA is a robust and independent predictor of prostate cancer recurrence. Common prognostic markers for prostate cancer (for example, such as a Gleason sum score) often fail to discriminate between the 10% of men with clinically detected prostate cancer who will die from their disease and the 90% that would not, even in the absence of treatment (Hahn and Roberts, J. Fam Practice 1993, 37:432-436; Lu-Yao et al., J.A.M.A., 1993, 269:2633; Fleming et al., J.A.M.A., 1993, 269:2650; Gerber et al., J.A.M.A., 19196, 276:615; Krongrad et al., J.A.M.A., 1997, 278:44; Partin et al., J.A.M.A., 1997, 277:1445; Epstein et al., Amer J. Surg. Path., 1996, 20:286; Drachenberg, Cancer Treat Rev. 2003, 29:235; and Drachenberg, Cancer Treat Rev. 2003, 29:231). Therefore, if TC in prostate biopsy, either alone or in combination with other prognostic markers, predicts with high sensitivity and specificity subsequent staging or the likelihood of disease-free survival, then men with prostate cancer will be able to make better-informed decisions about the potential risks and benefits of their treatment options. In this context, the independent relative risk for disease recurrence associated with TC values less than 0.75 (RH=5.02), was similar to those associated with moderately differentiated tumors with Gleason sum scores of 7 (RH=4.5), poorly differentiated tumors with Gleason sum scores of 8-9 (RH=5.96) or tumors with pelvic node involvement (RH=6.50). This finding is consistent with the hypothesis that phenotypic variability resulting from genomic instability caused by telomere dysfunction promotes the genesis of lethal, metastatic tumor cells. Moreover, the results confirm and extend the findings of our prior case-control study (Donaldson et al., J. Journal of Urology, 1999, 162:1788-92), which demonstrated a significant relationship between TC and prostate cancer recurrence and survival, thus providing additional support for the idea that TC could be a new marker for the prognosis of prostate cancer. Moreover, the surprising observations that TC is similar in tumor and CHN tissues, and that TC in DNA from CHN prostate tissue is also associated with prostate cancer recurrence, suggests that normal cells present in prostate biopsy specimens would not diminish the prognostic value of the TC assay, precluding the necessity of pure tumor samples or tissue microdissection, or the necessity to use cell-specific assays, such as in situ hybridization.

There is no a priori reason to believe that the relationships reported here between TC, genomic instability, phenotypic variability, and clinical outcome are unique to the prostate. For example, breast cancers are also glandular derived cancers in which steroid hormones drive proliferation and play a role in disease progression. Moreover, it has been previously shown that TC was associated with genomic instability, as defined by aneuploidy, in a random cohort of women with breast cancer (Griffith et al., Breast Cancer Research & Treatment, 1999, 54:59-64). Furthermore, multiple investigators have reported that genomic instability, as revealed by allelic imbalance occurs in CHN breast tissue (Forsti et al., Eur. J. Cancer 2001, 11:1372-1380; and Moinfar et al., Cancer Res. 2000, 60:2562-2566) and, in one report, was associated with the likelihood of breast cancer recurrence (Larson et al., Am. J. Pathol. 2002, 161:283-290). Finally, Meeker and coworkers recently reported that telomere shortening occurs in subsets of normal breast epithelium (Meeker et al., Am J. Pathol. 2004, 164:925-935), similar to what we have observed in prostatic CHN tissues. Subsequent studies with larger and more diverse patient populations are necessary to confirm and extend the relationship between TC and clinical outcome in these, and potentially other, tumor types and to determine the relationship between TC and other prognostic markers. Investigations of the prognostic value of TC in prostate biopsies are particularly important. However, the provocative findings of the current investigation clearly justify these studies.

In summary, this example demonstrates that defects in telomere maintenance in seemingly normal prostate cells create domains of tissues that are characterized by dysfunctional telomeres and increased genomic instability. Tumors developing in these domains are proposed to have greater phenotypic variability than tumors developing in fields with functional telomeres and more stable genomes, and thus are predicted to have the greatest probability of containing cells capable of invasion, extravasation and metastasis; i.e. those that will result in poor patient outcome. Such conclusions are consistent with a recent review by Bernards and Weinberg, who revisited the notion that some tumors are predisposed to aggressive metastatic phenotypes, even at inception (Bernards and Weinberg, Nature 2002, 418:823).

### Example 3

### Field Cancerization in Histologically Normal Tissue Adjacent to Breast Tumors as Determined by Telomere DNA Content and Allelic Imbalance

In this example, two markers of genomic instability, telemetric DNA content (TC) and allelic imbalance (AI), were measured in two independent sample sets of mammary carcinomas. This example demonstrates that telomere attrition and increased allelic imbalance not only occur in tumor specimens, but also in the surrounding tumor-adjacent, histologically normal tissues. Furthermore, the results of this example show that the extent of these genetic changes is a function of the distance from the visible tumor margin. These results are in agreement with the concepts of “field cancerization” and “cancer field effect,” terms that were previously introduced to describe areas within tissues consisting of histologically normal, yet genetically aberrant, cells that represent fertile grounds for tumorigenesis. The finding that markers of genomic instability occur in fields of histologically normal tissues is of practical importance, as it has implications for the identification of tumor margins, assessment of recurrence risk factors, and consideration of tissue-sparing surgery.

To better define the extent and spatial distribution of genomic instability in tissues adjacent to breast tumors, two independent, yet conceptually linked markers of genomic instability, TC and allelic imbalance, were measured in breast tumors and their matched histologically normal tumor-adjacent tissues. Towards this end, two different sample sets of breast cancer cases were used. The first samples were part of an archival cohort of breast cancer cases with tumor-adjacent tissues excised at unknown distances from the tumor margins, representing a scenario typical of retrospective studies using paraffinized tissue material. The second sample set represented a controlled and prospective study with tumor-adjacent tissues freshly excised at two defined distances, i.e. at 1 centimeter (cm) and 5 cm, from the visible tumor margins. Results obtained from the first retrospective sample confirm the findings of Example 2, a retrospective study performed in prostate cancer, which showed that telomere dysfunction occurs in disease-affected, yet histologically normal prostatic tissues. See also Fordyce et al., J Urol 2005, 173:610-614. In addition, the second sample set yielded better insights into the two-dimensional distribution of cells affected by genomic instability in tissues surrounding breast carcinomas. Overall, the results of the present example show that histologically normal breast tissues reflect the properties of their corresponding adjacent tumors, supporting the theory that fields of histologically normal, but genetically unstable, cells provide a fertile ground for tumorigenic events in breast tissues.

Materials and Methods

Breast Tissue Samples. Two independent collections of human breast tissues were used in this study. The first sample set was provided by the New Mexico Tumor Registry (NMTR) and consisted of 38 archival breast tumors and their matched histologically normal adjacent tissues from women who had undergone partial or full mastectomies between 1982 and 1993. The women ranged in age from 35 to 75 years, with a mean of 52, a median of 50, and a SD of 10.0 years. The tumors were typically large, had metastasized to the lymph nodes, and were grade 2 or 3. Adjacent tissues were excised at undefined distances from the tumor margins, yet originating from different tissue blocks. The second sample set consisted of eleven full mastectomy cases with tumors featuring clearly visible margins that were obtained from the University of New Mexico Health Sciences Center Pathology Laboratory. The women ranged in age from 26 to 61 years, with a mean of 44, a median of 53, and a SD of 11.2 years. The tumors included different grades, i.e. grade 1 (n=2), grade 2 (n=3), grade 3 (n=5), and ductal carcinoma in situ (n=1). Approximately 500 micrograms (μg) of tissue were excised from the tumors at both 1 cm and 5 cm from the visible tumor margins. After resection, the tissues were immediately frozen in liquid nitrogen. 10-12 μm sections were prepared and stained with hematoxylin and eosin by the Human Tissue Repository Service of the Department of Pathology. The sections were examined microscopically to assess their histological status. In addition, sections of the breast tumors were collected midway through the sectioning process, and stored at −70° C. until used for genomic DNA isolation. Twenty disease-free breast tissue samples from women undergoing reduction mammoplasty were obtained from the National Cancer Institute Cooperative Human Tissue Network (Nashville, Tenn.). The women ranged in age from 12 to 37 years, with a mean and median both of 27, and a SD of 7.9 years. All tissues used in this study were anonymous, and experiments were performed in accordance with all federal guidelines as approved by the University of New Mexico Health Science Center Human Research Review Committee.

Telomere DNA Content (TC) Assay. TC was measured using the slot blot titration assay as described by Fordyce et al., Biotechniques 2002, 33:144. Briefly, DNA was isolated using Qiagen DNeasy Tissue kits (Qiagen, Valencia, Calif.), denatured at 56° C. in 0.05 M NaOH/1.5 M NaCl, neutralized in 0.5 M Tris/1.5 M NaCl, and applied and UV cross-linked to Tropilon-Plus blotting membranes (Applied Biosystems, Foster City, Calif.). A telomere-specific oligonucleotide, end-labeled with fluorescein, (5′-TTAGGG-3′)₄-FAM, (IDT, Coralville, Iowa) was hybridized to the genomic DNA, and the membranes were then washed in wash buffers containing SSC and SDS of increasing stringency to remove non-hybridizing oligonucleotides. Hybridized oligonucleotides were detected by using an alkaline phosphatase-conjugated anti-fluorescein antibody that produces light when incubated with the CDP-Star substrate (Applied Biosystems, Foster City, Calif.). Blots were exposed to Hyperfilm for two to ten minutes (Amersham Pharmacia Biotech, Buckinghamshire, UK) and digitized by scanning. The intensity of the telomere hybridization signal was measured from the digitized images using Nucleotech Gel Expert Software 4.0 (Nucleotech, San Mateo, Calif.). TC is expressed as a percentage of the average chemiluminescent signal of three replicate tumor DNAs compared to the value of the placental standard of the same amount of genomic DNA (typically 20 ng). In addition to placental DNA, DNA purified from HeLa cells, which has approximately 30% of placental TC was frequently included to confirm the reproducibility of the assay.

Allelic Imbalance (AI) Assay. Approximately 1 ng of DNA was amplified using the AmpFISTR® Identifiler PCR Amplification Kit (Applied Biosystems, Foster City, Calif.) and following the manufacturer's protocol. This kit allows the amplification of 16 unlinked and genome-wide short tandem repeat (STR) microsatellite loci (Amelogenin, CSF1PO, D2S1338, D3S1358, D5S818, D7S820, D8S1179, D13S317, D16S539, D18551, D19S433, D21S11, FGA, TH01, TPOX and vWA) in a single multiplexed PCR reaction featuring four fluorescent dyes (6-FAM, PET, VIC and NED), each with a unique emission profile, thus allowing the simultaneous resolution of amplicons of 16 similar size. PCR products were resolved by capillary gel electrophoresis and detected using an ABI Prism 377 DNA Sequencer (Perkin Elmer, Foster City, Calif.). Data were analyzed using the ABI Prism GeneScan and Genotype Analysis software (Applied Biosystems, Foster City, Calif.). The height ratios of heterozygous alleles, indicated by two paired peaks, were calculated. By convention, the allele with the greater fluorescence intensity was used as the numerator. Thus the ratio was always greater than or equal to 1.0, with 1.0 representing the ratio for perfectly normal alleles. Using this approach, analysis of 318 heterozygous loci in buccal cells from 28 healthy individuals resulted in a mean ratio of 1.15 (SD=0.18). Based on these numbers, allelic pairs with a ratio of >1.61, i.e. the mean +2.5 SD, were scored as indicative of allelic imbalance.

Statistical Analysis. Linear regression analyses were performed using the JMP® statistical package (SAS Institute, Cary, N.C.) at a significance level of 0.05. The non-parametric Wilcoxon/Kruskal-Wallis Log Rank test was used to determine the correlation between markers of genomic instability (TC and AI), and spatial location, i.e. tumor, and histologically normal tissues, either at unknown tumor-adjacent locations (first sample set), or at 1 cm or 5 cm distance from visible tumor margins (second sample set).

Results

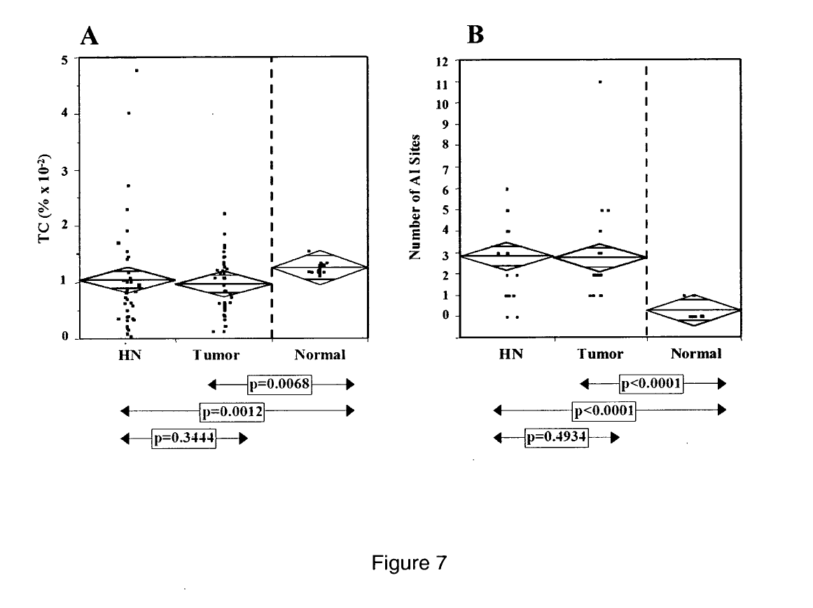

Telomeric DNA Content and Extent of Allelic Imbalance in Cancerous and Histologically Normal Breast Tissues. Previous studies in prostate and breast cancer tissues have shown that telomere DNA content (TC), a surrogate for telomere length (Fordyce et al., Biotechniques 2002, 33:144; and Bryant et al., Biotechniques 1997, 23:476), is a prognosticator of recurrence and clinical outcome in prostate cancer (Donaldson et al., J Urol 1999, 162:1788-1792; Meeker et al., Cancer Res 2002, 62:6405-6409; and Fordyce et al., J Urol 2005, 173:610-614), and of genomic instability and metastasis in invasive human breast carcinomas (Griffith et al., Breast Cancer Res Treat 1999, 54:59-64). As shown in Example 2, TC in histologically normal tissues adjacent to prostate tumors also predicts the course of disease. See also Fordyce et al., J Urol 2005, 173:610-614. In analogy to the latter studies performed using tissues from patients with prostate adenocarcinomas, the present example extends this analysis to an independent cohort of breast cancer cases consisting of 38 archival breast tumors and their matched histologically normal adjacent tissues from women who had undergone partial or full mastectomies between 1982 and 1993. These tumors were typically large, had metastasized to the lymph nodes, and were grade 2 or 3. Corresponding adjacent, histologically normal tissues were excised at undefined distances from the tumor margins. To define the normal range of TC in breast tissues not affected by disease, TC was first measured in breast tissues obtained from 20 women undergoing reduction mammoplasty. These measurements defined a normal range of TC of 114% to 158%, with a mean of 127% and a median of 125% of placental control (FIG. 7A). In contrast, the tumors and matched histologically normal adjacent tissues showed a widely distributed range of TC, i.e. 14%-224% (mean 98%, median 102%) and 6%-480% (mean 105%, median 85%), respectively (FIG. 7A). Importantly, these measurements indicated that TC in both tumor and matched histologically normal adjacent tissues was different from TC in normal disease-free breast tissues (p=0.0068 and p=0.0012, respectively), whereas TC in tumors and matched histologically normal adjacent tissues showed a similar distribution (p=0.3444), indicating both shortening and lengthening of telomeres in both types of tissue derived from the breast cancer patients (FIG. 7A).

Since telomere attrition induces genomic instability (Desmaze et al., Cancer Lett 2003, 194:173-182; Callen and Surralles, Mutat Res 2004, 567:85-104; Hackett et al., Cell 2001, 106:275-286; and Mathieu et al., Cell Mol Life Sci 2004, 61:641-656), these results were corroborated by investigating an independent marker of genomic instability as measured by the extent of allelic imbalance (AI) at 16 genome-wide microsatellite regions. These measurements were performed in 23 of the 38 samples presented in FIG. 7A. The mean number of sites affected by allelic imbalance in the disease-free breast tissue samples, tumors and in the matched histologically normal tissues were 0.3, 2.73, and 2.82, respectively (FIG. 7B). As with TC, the extent of allelic imbalance in both tumor and histologically normal tissues was significantly different from that observed in disease-free breast tissues (p<0.0001 for both), whereas the extent of allelic imbalance was similar in the tumor and matched histologically normal tissues (p=0.4934). Together, the measurements of TC and allelic imbalance in breast tissues from breast cancer patients suggest that generally, tissues adjacent to breast carcinomas, although histologically normal in appearance, is genetically altered.

Histology of Cancerous and Histologically Normal Breast Tissues. The latter result prompted studies to determine the field size of genomic instability in histologically normal tumor-adjacent breast tissues. Towards this end, 11 breast tumor samples, derived from women undergoing full mastectomies, and their corresponding matched adjacent tissues, excised at 1 cm and 5 cm from the visible tumor margins, were histologically examined after staining with hematoxylin and eosin. Sections of the tumors demonstrated abnormal architecture with fields of infiltrating ductal carcinoma and ductal carcinoma in situ. Histological examination of the tumor-adjacent tissues at 1 cm and 5 cm from the visible tumor margins indicated normal breast tissue architecture with normal lobular units and ducts, as well as adipose tissue. Overall, these tumors included different grades, i.e. grade 1 (n=2), grade 2 (n=3), grade 3 (n=5), and ductal carcinoma in situ (n=1).

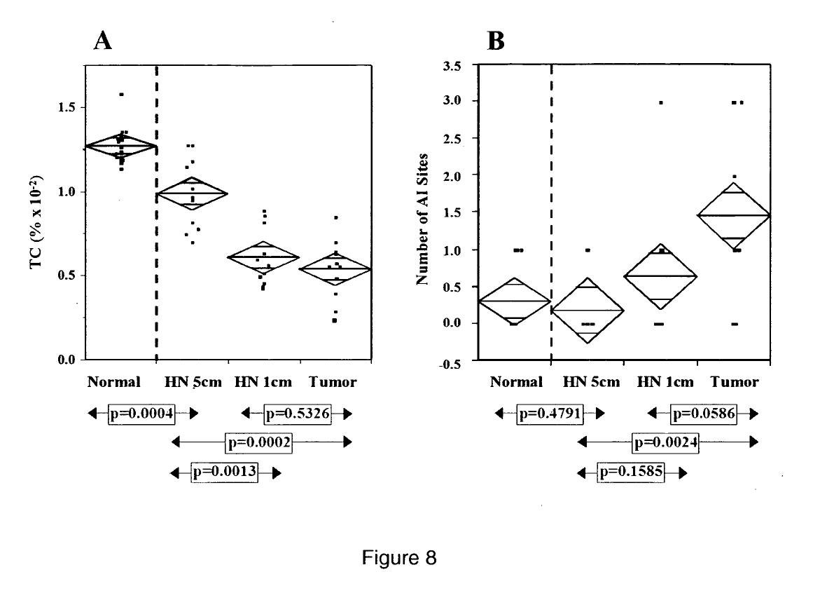

Field Effect of Genomic Instability in Breast Cancer Tissue. Next, the spatial distribution of markers of genomic instability was determined in the 11 breast cancer cases and their corresponding histologically normal, adjacent tissues, excised at 1 cm and 5 cm from the visible tumor margins, by measuring TC and extent of allelic imbalance. As with the first sample set, these findings were compared with TC and allelic imbalance measured in the 20 normal, disease-free breast tissues obtained from reduction mammoplasty (see FIG. 7). As shown in FIG. 8A, the mean and the median TC in tumors were 54% and 56% of placental control, respectively. The mean and median TC values in the histologically normal adjacent tissues were 61% and 57% at 1 cm, and 99% and 97% at 5 cm from the visible tumor margins, respectively. In agreement with previous studies, the lowest values for TC were observed in the tumor tissues. In addition, while TC in the tumors and at 1 cm from the visible tumor margin were similar (p=0.5326), TC at 5 cm from the visible tumor margin was different from TC in tumors (p=0.0002), as well as from TC at the 1 cm site (p=0.0013). Finally, the data obtained in this example suggest a smaller, yet significant difference between TC in normal, disease-free breast tissues and histologically normal tissues excised at 5 cm from the visible tumor margin (p=0.0004). The difference in TC distribution observed in the HN tissues of the two sample sets (FIG. 7A vs. 8A) are most probably due to the either the fact that the 14N tissue in the first set was removed at an undefined distance, or to the different storage method of the tissue (paraffinized vs. frozen). Overall, the data in FIG. 8A shows that TC increases as a function of distance from the visible tumor margin.

As with the first sample set, and again as a confirmation of the genomic instability induced by telomere attrition, the extent of allelic imbalance was determined in the 11 breast cancers and their matched tumor-adjacent tissues. Analysis of a total of 137 heterozygous genomic loci revealed the highest level of allelic imbalance in the tumors, with a mean of 1.45 sites affected by AI (FIG. 8B). The mean number of sites affected by allelic imbalance in the histologically normal adjacent tissues was 0.63 at 1 cm, and 0.18 at 5 cm from the visible tumor margins, respectively. The extent of allelic imbalance in the tumor and histologically normal tissues excised at 5 cm from the visible tumor margins were significantly different (p=0.0024). This analysis further indicated a marginal difference between the extent of allelic imbalance in the tissues removed at 1 cm from the visible tumor margins and extent of allelic imbalance in the tumor (p=0.0586), while allelic imbalance in HN at 1 and 5 cm was similar (p=0.1585). Overall, the mean of allelic imbalance did follow an expected trend as a function of distance from the visible tumor margin (FIG. 8B). Interestingly, and in contrast to observations with regard to TC, the extent of allelic imbalance in normal disease-free breast tissues was similar to allelic imbalance in histologically normal breast tissues excised at 5 cm from breast cancer margins (p=0.4791). Nevertheless, the present studies on allelic imbalance again indicate an overall decrease of genomic instability with increasing distance from the visible tumor margin. Finally, it should be noted that the difference in the number of genomic sites affected by allelic imbalance in the two sample sets (FIG. 7B vs. FIG. 8B) could be explained either by differences in tumor types, or by type and length of tissue storage, i.e. paraffinized vs. frozen.

Extent of Conservation of Allelic Imbalance between Tumor and Adjacent Tissues. To address the question of whether locus-specific allelic imbalance is part of the widely accepted concept of clonal evolution of cancer, the frequency of imbalance for each measured allele in the two sample sets was determined, as well as the extent of conservation of affected alleles between tumor and histologically normal tissue (Table 4). These results show that 11 of the analyzed genomic loci were conserved in 4.3%-17.4% of the cases in the first sample set, and that only 2 genomic loci were conserved in 9.1%-18.2% of the cases in the second sample set.

TABLE 4
Frequencies (in %) of measured imbalances for each genomic locus, and of
imbalances conserved between tumor and histologically normal tissues.

| Genomic | D8S1 | D21S | D7S8 | CSF | D3S1 | TH0 | D13S | D16S | D2S1 | D19S | | TPO | D18S | Am- | D5S8 | |
|---|---|---|---|---|---|---|---|---|---|---|---|---|---|---|---|---|
| Locus | 179 | 11 | 20 | 1PO | 358 | 1 | 317 | 539 | 338 | 433 | vWA | X | 51 | X | 18 | FGA |
| 1^{st} Set^{(a)} | | | | | | | | | | | | | | | | |
| Tumor | 34.8 | 39.1 | 17.4 | 4.3 | 26.1 | 17.4 | 21.7 | 21.7 | 4.3 | 17.4 | 17.4 | 4.3 | 4.3 | 0.0 | 17.4 | 21.7 |
| HN^{(c)} | 17.4 | 26.1 | 13.0 | 4.3 | 21.7 | 17.4 | 30.4 | 30.4 | 17.4 | 21.7 | 34.8 | 8.7 | 13.0 | 0.0 | 8.7 | 21.7 |
| Conserved^{(c)} | 13.0 | 17.4 | 4.3 | 0.0 | 0.0 | 4.3 | 13.0 | 4.3 | 4.3 | 8.7 | 8.7 | 0.0 | 0.0 | 0.0 | 4.3 | 8.7 |
| 2^{nd} Set^{(b)} | | | | | | | | | | | | | | | | |
| Tumor | 18.2 | 9.1 | 0.0 | 0.0 | 9.1 | 0.0 | 18.2 | 9.1 | 9.1 | 9.1 | 36.4 | 0.0 | 18.2 | 0.0 | 0.0 | 9.1 |
| HN^{(c)} 1 cm | 0.0 | 0.0 | 0.0 | 0.0 | 0.0 | 0.0 | 9.1 | 18.2 | 0.0 | 0.0 | 9.1 | 9.1 | 0.0 | 0.0 | 9.1 | 0.0 |
| HN^{(c)} 5 cm | 0.0 | 0.0 | 0.0 | 0.0 | 0.0 | 0.0 | 0.0 | 0.0 | 0.0 | 0.0 | 9.1 | 9.1 | 0.0 | 0.0 | 0.0 | 0.0 |
| Conserved^{(d)} | 0.0 | 0.0 | 0.0 | 0.0 | 0.0 | 0.0 | 0.0 | 9.1 | 0.0 | 0.0 | 18.2 | 0.0 | 0.0 | 0.0 | 0.0 | 0.0 |

^{(a)}n = 23 paraffinized cases;
^{(b)}n = 11 frozen cases;
^{(c)}HN, histologically normal;
^{(d)}conserved in either HN 1 cm or HN 5 cm as compared with the tumor.

In addition, only two loci, i.e. D16S539 and vWA, showed conservation between histologically normal tissues and the corresponding tumors in both sample sets.

Nevertheless, the collective data of this example indicate that histologically normal breast tissues contain properties of their proximal tumors with regard to markers of genomic instability, such as telomere length and allelic imbalance. Also, the results presented in this example suggest the presence of a “field of increasing genomic instability” as a function of distance from mammary carcinomas.

Discussion

Although mechanistic insights into the molecular pathology of sporadic breast cancers are increasing (Lakhani, Mol Pathol 2001, 54:281-284), the question of how carcinogenesis is initiated in human breast tissues remains largely unanswered (Kenemans et al., Maturitas 2004, 49:34-43). It is, however, widely accepted that genomic instability is a prerequisite of virtually all tumors, including breast cancers, facilitating the accumulation of further genetic alterations responsible for cancer progression (Gollin, Curr Opin Oncol 2004, 16:25-31; Charames and Bapat, Curr Mol Med 2003, 3:589-596; Nojima, Methods Mol Biol 2004, 280:3-49; and O'Connell, Breast Cancer Res Treat 2003, 78:347-357). It has also been shown that shortened telomeres increase the mutation rate and the genomic instability of affected cells (Callen and Surralles, Mutat Res 2004, 567:85-104; and Hackett et al., Cell 2001, 106:275-286).

An important observation in this study is that genomic instability occurs in histologically normal breast tissues adjacent to the corresponding tumors. A further, major observation of this study is the finding that the extent of this genomic instability in histologically normal tumor adjacent breast tissues is a function of distance from the visible tumor margins, and that it affects a rather large field surrounding breast carcinomas. In this regard, two independent quantitative measures of instability were assessed; telomeric DNA content (TC) and allelic imbalance (AI). The results obtained are in general agreement with the work of previous investigators who reported that genetic alterations, including telomere attrition and loss of heterozygosity, occur in histologically normal tissues adjacent to breast tumors (Aubele et al., Diagn Mol Pathol 2000, 9:14-19; Farabegoli et al., J Pathol 2002, 196:280-286; Deng et al., Science 1996, 274:2057-2059; Forsti et al., European J Cancer 2001, 37:1372-1380; Lakhani et al., Journal of Pathology 1999, 189:496-503; Larson et al., Am J Pathol 2002, 161:283-290; Meeker et al., Am J Pathol 2004, 164:925-935; Euhus et al. Journal of the National Cancer Institute 2002, 94:858-860; and Ellsworth et al., Breast Cancer Res Treat 2004, 88:131-139). In these previous studies, the sites of telomere attrition, loss of heterozygosity, and allelic imbalance were physically distant from one another and from the tumors, albeit at undefined distances from the corresponding tumor lesions in most cases (Hirashima et al., Anticancer Research 2000, 20:2181-2187; Meeker et al., Am J Pathol 2004, 164:925-935; Euhus et al. Journal of the National Cancer Institute 2002, 94:858-860; and Ellsworth et al., Breast Cancer Res Treat 2004, 88:131-139). In contrast, the present example is the first study that analyzes genomic instability in breast cancers at two different and defined distances, at 1 cm and 5 cm from the visible tumor margins. This study indicates a field of genomic instability harboring less genetic alterations with increasing distance from the tumor lesion. The present example also supports the concept that telomere attrition induces genomic instability in human tumors (Desmaze et al., Cancer Lett 2003, 194:173-182; Callen and Surralles, Mutat Res 2004, 567:85-104; and Hackett et al., Cell 2001, 106:275-286). The latter is shown by the fact that TC was different between disease-free breast tissues and tumor adjacent tissues excised at 5 cm from the tumor margin, while the extent of allelic imbalance was not, thus implying that telomere attrition precedes the onset of allelic imbalance. Finally, while the results on the low frequency of conservation of alleles affected by imbalance between tumors and the corresponding adjacent tissues could be due to the use of microsatellite markers that are unlinked to breast cancer, these observations are in agreement with previous studies by Larson and colleagues that showed that loss of heterozygosity and allele imbalance in histologically normal breast epithelium is distinct from loss of heterozygosity or allele imbalance in co-existing carcinomas (Larson et al., Am J Pathol 2002, 161:283-290).

The present study supports a theory that breast epithelial carcinogenesis occurs at higher frequency in fields of increased genomic instability. This is supported by observations that two independent markers of genomic instability, telomere attrition and extent of allelic imbalance, converged to be highest in the tumor lesions and gradually decreased with increasing distance from the tumor. The present results support the concept of “field effect cancerization,” introduced by Slaughter and colleagues in 1953 (Slaughter et al., Cancer 1953, 6:963-968) and reviewed by others (Braakhuis et al., Cancer Res 2003, 63:1727-1730; and Garcia et al., J Pathol 1999, 187:61-81). These authors developed this term to explain the multifocal and independent areas of histologically pre-cancerous alterations occurring in oral squamous cell carcinomas (Slaughter et al., Cancer 1953, 6:963-968). Organ systems in which field cancerization has been implied include lung, colon, cervix, bladder, skin, and breast (Hockel and Dornhofer, Cancer Res 2005, 65:2997-3002). The concept of cancerization has also been used to explain the occurrence of genetic and epigenetic mosaicism in cancer precursor tissues (Tycko, Ann N Y Acad Sci 2003, 983:43-54). The present example extends this concept to genetic alterations in otherwise histologically normal tissues and is the first to propose to include telomere attrition.

Fields of genomic instability that support tumorigenic events have important clinical implications. First, such fields could give rise to clonal selection of precursor cells that ultimately lead to the development of breast cancer (Ellsworth et al., Lancet Oncol 2004, 5:753-758). Second, the presence of such fields, even after surgical resection of primary tumors, would represent a continuous risk factor for cancer recurrence or formation of secondary lesions (Garcia et al., J Pathol 1999, 187:61-81; and Li et al., Cancer Res 2002, 62:1000-1003). For example, in head and neck squamous carcinogenesis, such fields have been estimated to be up to 7 cm in diameter (Braakhuis et al., Semin Cancer Biol 2005, 15:113-120). In agreement with these studies, the results of the present example indicate the occurrence of telomere attrition in breast tissues as distant as 5 cm from the visible tumor margins.

The present study is of clinical importance and directly addresses the assessment of tumor margins for breast cancer surgical procedures. In this regard, the genetically altered field surrounding breast tumors could constitute a risk for local recurrence, which happens in up to 22% of patients undergoing breast conservation therapies for small invasive and non-invasive breast cancers (Huston and Simmons, Am J Surg 2005, 189:229-235). Other aspects of breast cancer affected by our findings include secondary treatment options, and prognosis (Klimberg et al., Surg Oncol 1999, 8:77-84). Finally, a better definition, as well as detection, of genetically altered fields within histologically normal breast tissues adjacent to tumor lesions may allow a better risk assessment for the development of lesions in the contra-lateral breast (Singletary, Am J Surg 2002, 184:383-393; and Meric-Bernstam, Curr Opin Obstet Gynecol 2004, 16:31-36). It is obvious that patients diagnosed with extensive fields of genomic instability may need a different follow-up, for example as characterized by more frequent and more focused screening (Meric-Bernstam, Cun Opin Obstet Gynecol 2004, 16:31-36). In summary, the results of the present example indicate that evaluation of surgical margins should be complemented by molecular genetic, rather than only histological techniques.

### Example 4

### A Simple, High-Throughput Method for Measuring the Extent of Genomic Instability in Tissue Samples

Example 1 describes an assay for quantitatively determining the extent of allelic imbalance (AI) in tissue samples. As shown in Examples 2 and 3, allelic imbalance, an indicator of genomic instability, has diagnostic and prognostic value in breast and prostate cancer. In addition, allelic imbalance in histologically normal tissue adjacent to the tumor displays similar genetic alterations and also has diagnostic and prognostic value.

Buccal cells were collected from the oral rinses of randomly selected volunteers, independent of gender or age. Frozen renal tissues, including both renal cell carcinomas and, in most instances, matched normal tissue, were obtained from radical nephrectomies and provided by the Cooperative Human Tissue Network. Two independent sets of breast archival invasive breast tumors, one comprised of frozen tissues and another of formalin-fixed, paraffin-embedded tissues with matched normal tissue, both obtained from lumpectomies or radical mastectomies, were provided by the University of New Mexico Solid Tumor Facility and New Mexico Tumor Registry Tissue Acquisition Service (NMTR-TAS), respectively. Breast tumors with matched normal tissue from 1 cm and 5 cm away were collected from the Department of Pathology at the University of New Mexico Hospital. In addition, the breast tumors with matched peripheral blood lymphocytes were obtained from the HEAL study at the University of New Mexico Hospital. Formalin-fixed, paraffin-embedded archival prostate tissues, obtained from radical prostatectomies, and matched biopsy tissue, were also provided by the NMTR-TAS. Two independent sets of matched prostatectomy and biopsy material were obtained from the Department of Internal Medicine, University of New Mexico and from the NCI Cooperative Prostate Cancer Tissue Resource, respectively. Finally, endometrial tumors were obtained from the Department of Obstetrics and Gynecology at the University of New Mexico Hospital. All specimens lacked patient identifiers and were obtained in accordance with all federal guidelines as approved by the University of New Mexico Human Research Review Committee. DNA isolation and quantification and multiplex PCR amplification of STR Loci were as described for Examples 1-3

The frequency of allelic ratios greater than 1.61 in DNA purified from 72 normal tissues comprised of buccal, renal and breast tissue was determined. 76% of the normal tissues did not display a site of allelic imbalance, 22% displayed one site of allelic imbalance and 1% displayed 2 sites or greater of allelic imbalance. In contrast, 90 coexisting histologically normal (CHN) tissues comprised of breast and prostate tissue were analyzed. 26% of the CHN tissues displayed no allelic imbalance, 22% displayed 1 sited of allelic imbalance and 52% displayed 2 sites or greater of allelic imbalance. In addition, 314 tumors comprised of breast, prostate, renal and endometrial tumors were analyzed. Only 12% of the tumor tissues displayed no sites of allelic imbalance; whereas 23% displayed 1 site of allelic imbalance and 65% of the tumor tissues displayed 2 or more sites of allelic imbalance. This data is shown in Table 5.

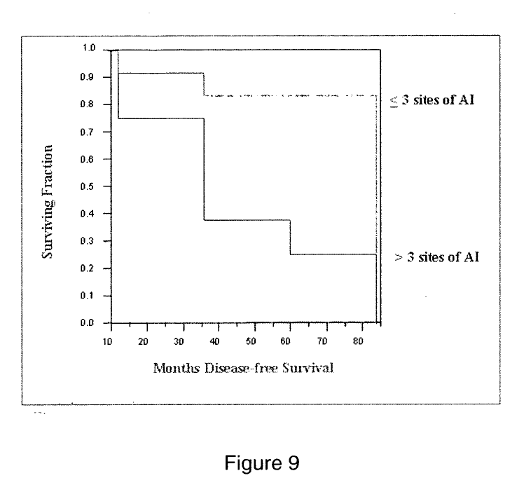

To determine the extent to which allelic imbalance would predict clinical outcome in breast cancer, breast tumors (n=31) were analyzed for allelic imbalance and divided the specimens into two groups based on the number of sites of allelic imbalance. As shown in FIG. 9, a significantly greater fraction of patients with less that three sites had increased disease-free survival when compared to the patients with greater than or equal to three sites of allelic imbalance (p=0.018).

TABLE 5
Distribution of number of sites of allelic imbalance in normal,
coexisting histologically normal (CHN) and tumor tissues. The three
tissue types are divided into groups based on the number of sites of
allelic imbalance (0, 1, ≧2).

| | 0 | 1 | ≧2 |
|---|---|---|---|
| Normal Tissue | | | |
| Buccal Tissue | 28 | 9 | 1 |
| Normal Renal Tissue | 9 | 1 | 0 |
| Normal Breast Tissue | 9 | 2 | 0 |
| PBLs | 9 | 4 | 0 |
| CHN Tissue | | | |
| CHN Breast Tissue | 8 | 7 | 12 |
| CHN Prostate Tissue | 15 | 13 | 35 |
| Tumor Tissue | | | |
| Breast Tumor Tissue | 8 | 17 | 58 |
| Prostate Tumor Tissue | 13 | 26 | 70 |
| Renal Tumor Tissue | 4 | 5 | 15 |
| Endometrial Tumor Tissue | 3 | 3 | 2 |

### Example 5

### Telomere DNA Content in Cancerous and Proximal Histologically Normal Tissues Predicts Disease-Free Survival in Breast Cancer Patients

Telomeres are specialized nucleoprotein complexes that protect and stabilize the ends of linear chromosomes. Telomere attrition, induced for example by incomplete DNA replication during mitosis, is a prime source of genomic instability and a hallmark of cancers, including breast cancer. Because genomic instability leads to phenotypic variability, which in turn drives the development of aggressive cell clones, this example demonstrates that telomeric DNA content (TC) predicts clinical outcome in breast cancer patients.

The present example included two independent cohorts of breast cancer specimens. The first cohort (n=25) was a case-control group consisting of large, node-positive tumors; for this group, proximal histologically normal (PHN) tissue was also available. The second cohort (n=54) consisted of randomly selected invasive ductal carcinomas. TC was measured using a chemiluminescence hybridization assay, following procedures described in more detail in Examples 2 and 3. Allelic imbalance (AI) was determined by multiplex PCR analysis of 16 genome-wide unlinked microsatellites, following procedures described in more detail in Examples 1-3. Associations between TC and either AI or disease recurrence were analyzed by Wilcoxon/Kruskal Wallis Rank Sums test; associations between TC and time of disease-free survival were determined by Kaplan/Meier Log Rank analysis.

The extent of AI was associated with TC in both breast cancer study groups (p=0.012 and p=0.05). In the first group, TC was associated with disease recurrence within 84 months of surgery (p=0.012) and with time of disease-free survival (p=0.017). TC was also associated with recurrence status and time of disease-free survival in PHN tissue (p=0.025 and p=0.006). Cases in the second cohort were stratified by mean TC; in this group TC was able to predict disease recurrence with a sensitivity of 87%.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

1. A method of detecting allelic imbalance in genomic nucleic acid, the method comprising:
coamplifying in a single reaction mixture a plurality of short tandem repeat (STR) loci in the genomic nucleic acid; wherein the STR loci are unlinked; and wherein each allele of each different STR locus yields an amplicon product;
detecting the resultant amplicon products; and
calculating an allelic ratio of the resultant amplicon products for each STR locus;
wherein a statistically significant allelic ratio of greater than 1.0 for a STR locus indicates an allelic imbalance at the STR locus.

2. The method of claim 1, wherein detecting the resultant amplicon products is by electrophoretic separation.

3. The method of claim 1, wherein detecting the resultant amplicon products is by mass spectrometry.

4. The method of claim 1, wherein coamplifying and detecting the resultant amplicon products is carried out in a single preparation.

5. The method of claim 1, wherein coamplifying and detecting the resultant amplicon products is carried out in more than one preparation.

6. The method of claim 1, wherein the allelic ratio is 1.28 or greater.

7. The method of claim 1, wherein the allelic ratio is 1.37 or greater.

8. The method of claim 1, wherein the allelic ratio is 1.61 or greater.

9. The method of claim 1, wherein the allelic ratio is 2.15 or greater.

10. The method of claim 1, wherein the genomic nucleic acid comprises genomic nucleic acid obtained from tumor cells.

11. The method of claim 1, wherein at least 12 different STR loci are amplified.

12. The method of claim 1, wherein at least 16 different STR loci are amplified.

13. The method of claim 1 wherein three or more STR loci exhibit allelic imbalance.

14. The method of claim 1, wherein one or more of the STR loci amplified are selected from the group consisting of amelogenin, CSF1PO, D2S1338, D3S1358, D5S818, D7S820, D8S1179, D13S317, D16S539, D18S51, D19S433, D21S11, FGA, TH01, TPOX, and vWA.

15. The method of claim 1, wherein the STR loci amplified comprise amelogenin, CSF1PO, D2S1338, D3S1358, D5S818, D7S820, D8S1179, D13S317, D16S539, D18S51, D19S433, D21 S11, FGA, TH01, TPOX, and vWA.

16. The method of claim 1, wherein the genomic nucleic acid comprises genomic nucleic acid obtained from histologically normal cells adjacent to a tumor.

17. A method of determining cancer prognosis, the method comprising:
coamplifying in a single reaction mixture a plurality of short tandem repeat (STR) loci in a genomic nucleic acid sample from histologically normal, tumor-adjacent tissue;
wherein the STR loci are unlinked; and wherein each allele of each different STR locus yields an amplicon product;
detecting the resultant amplicon products; and
calculating an allelic ratio of the resultant amplicon products for each STR locus;
wherein a statistically significant allelic ratio of greater than 1.0 for a STR locus indicates an allelic imbalance at the STR locus; and
wherein an allelic imbalance in at least one STR locus is indicative of a cancer with an increased risk for metastasis, recurrence and/or death.

18. The method of claim 17, wherein three or more STR loci are amplified and wherein an allelic imbalance in at least three STR loci is indicative of a cancer with an increased high risk for metastasis, recurrence and/or death.

19. A method of identifying a tumor margin, the method comprising:
coamplifying in a single reaction mixture a plurality of short tandem repeat (STR) loci in a genomic nucleic acid sample from tumor-adjacent tissue; wherein the STR loci are unlinked; and wherein each allele of each different STR locus yields an amplicon product;
detecting the resultant amplicon products; and
calculating an allelic ratio of the resultant amplicon products for each STR locus;
wherein a statistically significant ratio of greater than 1.0 for a STR locus indicates an allelic imbalance at the STR locus; and
wherein an allelic imbalance in at least one STR locus identifies the tumor-adjacent tissue as within the margin of the tumor.

20. The method of claim 19, wherein three or more STR loci are amplified, and wherein an allelic imbalance in at least three STR loci identifies the tumor-adjacent tissue as within the margin of the tumor.

21. A method of diagnosing cancer, the method comprising:
coamplifying in a single reaction mixture a plurality of short tandem repeat (STR) loci in a genomic nucleic acid sample; wherein the STR loci are unlinked; and wherein each allele of each different STR locus yields an amplicon product;
detecting the resultant amplicon products; and
calculating an allelic ratio of the resultant amplicon products for each STR locus;
wherein a statistically significant allelic ratio of greater than 1.0 for a STR locus indicates an allelic imbalance at the STR locus; and
wherein an allelic imbalance in at least one STR locus indicates that the sample includes cancerous cells.

22. The method of claim 21, wherein three or more STR loci are amplified, and wherein allelic imbalance in at least three STR loci indicates that the sample includes cancerous cells.

23. A method of identifying a predisposition to cancer, the method comprising:
coamplifying in a single reaction mixture a plurality of short tandem repeat (STR) loci in a genomic nucleic acid sample from an individual with a suspected predisposition to cancer; wherein the STR loci are unlinked; and wherein each allele of each different STR locus yields an amplicon product;
detecting the resultant amplicon products; and
calculating an allelic ratio of the resultant amplicon products for each STR locus;
wherein a statistically significant allelic ratio of greater than 1.0 for a STR locus indicates an allelic imbalance at the STR locus; and
wherein an allelic imbalance in at least one STR locus indicates that the subject has a predisposition to cancer.

24. The method of claim 23, wherein three or more STR loci are amplified, and wherein allelic imbalance in at least three STR loci indicates that the subject has a predisposition to cancer.
